(19) United States
(12) Patent Application Publication
(10) Pub. No.: US 2019/0175502 A1
(43) Pub. Date: Jun. 13, 2019

(54) METHODS AND COMPOSITIONS FOR TREATING LUNG DISEASE OF PREMATURITY

(71) Applicant: INDIANA UNIVERSITY RESEARCH & TECHNOLOGY CORPORATION, Indianapolis, IN (US)

(72) Inventors: Margaret Arlene Schwarz, III, Bristol, IN (US); Dong II Lee, Indianapolis, IN (US)

(21) Appl. No.: 15/768,994

(22) PCT Filed: Oct. 20, 2016
(86) PCT No.: PCT/US2016/057900

§ 371 Date: Apr. 17, 2018

Related U.S. Application Data
(60) Provisional application No. 62/243,813, filed on Oct. 20, 2015.

(51) Int. Cl. A61K 9/00; C07K 16/24; A61K 39/395; A61K 33/00; A61K 31/56; A61P 11/00

(52) U.S. Cl. CPC A61K 9/0078; C07K 16/24; A61K 39/3955; A61K 33/00; A61K 2039/505; A61K 9/007; A61P 11/00; A61K 9/0082; A61K 31/56

(57) ABSTRACT

The disclosure relates to methods of treating an infant having or at risk of developing bronchopulmonary dysplasia, including premature infants, by administering an antagonist of endothelial monocyte-activating polypeptide II (EMAP II) to the infant.

### CROSS-REFERENCE TO RELATED APPLICATIONS

This application claims priority to U.S. Provisional Application No. 62/243,813 filed on Oct. 20, 2015, the contents of which are incorporated by reference in its entirety.

### STATEMENT REGARDING FEDERALLY SPONSORED RESEARCH AND DEVELOPMENT

This invention was made with government support under R01 HL114977 awarded by the National Institutes of Health. The government has certain rights in the invention.

### BACKGROUND

Lung disease of prematurity is among the disease states driven by inflammation. Placed on supportive care, prematurely born children with underdeveloped lungs commonly progress toward development of chronic lung disease, specifically bronchopulmonary dysplasia (BPD). Currently, premature birth is the leading cause of death in children under the age of five affecting 1 in 10 births and representing approximately 15 million births per year worldwide (1-4). In its most severe form, BPD can result in secondary cardiovascular sequelae such as pulmonary hypertension (PH) that persist into adulthood and abnormal ventilatory response (5-10). Despite advances in clinical ventilator management, the introduction of surfactant, and antenatal glucocorticoids, there is a marked lack of adjunctive therapies.

Pulmonary inflammation significantly contributes to the multifactorial pathogenesis of BPD (11-15). Like other lung injuries that are driven by inflammation such as asthma, in BPD, bronchial epithelial cells and myeloid cells with macrophage lineage are key effectors driving the secretion of both cytokines and chemokines such as IL-1β and MCP-1, respectively.

Clinically, current therapies administered to the premature infants from birth include either surfactants to aid alveolar plasticity or glucocorticoids to limit inflammation and thereby to prevent BPD progression in premature infants. As expected, tracheal aspirates of infants exposed to hyperoxia had elevated inflammatory mediators primarily secreted by macrophages, notably IL-1β and TNF-α (14, 15). In infants with sepsis-induced inflammation, inhibitors against the two cytokines showed little improvement in survival rates; in mouse models treated with inhibitors against these cytokines, only some BPD features improved (16-20). This suggests that alternative, more broadly functioning or upstream targets are needed to prevent BPD.

In BPD, studies have identified candidate cytokines to be predictive of BPD onset. However, the source, function, and physiological mechanisms that drive the inflammatory state are poorly understood.

Despite the advent of systemic surfactant and anti-inflammatory medication, the number of preterm infants diagnosed with BPD continues to rise. Accordingly, there remains a need in the field for targeted therapeutic methods to minimize inflammation while promoting normal alveolar formation.

### SUMMARY OF THE INVENTION

In a first aspect, provided herein is a pharmaceutical composition comprising or consisting essentially of a therapeutically effective amount of an antagonist of endothelial monocyte-activating polypeptide II (EMAP II), and a pharmaceutically suitable carrier. The antagonist of EMAP II can be selected from the group consisting of an anti-EMAP II antibody, an antibody specific for an EMAP II receptor, and a soluble EMAP II receptor.

In another aspect, provided herein is a method of treating a lung condition in a subject, in need thereof. The method comprises administering to the subject a therapeutically effective amount of a pharmaceutical composition comprising a pharmaceutically effective amount of an antagonist of EMAP II and a pharmaceutically suitable carrier, whereby the lung condition is treated in the subject. In some aspects, the subject is an infant, and in some instances a neonate. The pharmaceutical composition can be used to ameliorate bronchopulmonary dysplasia in an infant that has been diagnosed with the lung condition. In some cases, the method further comprises administering at least one additional agent or therapy selected from the group consisting of a surfactant, oxygen therapy, ventilator therapy, steroid, or inhaled nitric oxide.

In another aspect, provided herein is a method of treating an infant at risk of developing bronchopulmonary dysplasia. The method comprises administering a therapeutically effective amount of a pharmaceutical composition comprising a therapeutically effective amount of an antagonist of EMAP II and a pharmaceutically suitable carrier to the infant. In some cases, the method further comprises administering at least one additional agent or therapy selected from the group consisting of a surfactant, oxygen therapy, ventilator therapy, steroid, or inhaled nitric oxide.

In yet another aspect, provided herein is a method of reducing macrophage infiltration into the lungs of a subject suffering from bronchopulmonary dysplasia. The method comprises administering a therapeutically effective amount of the pharmaceutical composition comprising a therapeutically effective amount of an antagonist of EMAP II to reduce the number of macrophage infiltrating into the lung of the subject.

In another aspect, provided herein is a use of the pharmaceutical composition of the present invention for treatment of a lung condition in a subject.

### BRIEF DESCRIPTION OF THE DRAWINGS

FIG. 1. EMAP II secreted by airway conducting epithelial cells of BPD mice recruit macrophages. (A) Experimental schematic of exposure of neonatal mice to oxygen to induce BPD. (B) EMAP II protein expression and (C) quantification in whole-lung lysates of normoxic and hyperoxic mice (normalized to β-actin, pooled samples of at least n=3 for day 3, n=2 for day 30, n=3-4 for day 10, n=6-10 for other days, at least two independent experiments). Main effect of oxygen, p=0.0000322, interaction of oxygen: Age, p=0.788. (D) Representative images of immunohistochemical (IHC) co-staining for EMAP II expression (red) and CCSP (green). Purple indicates co-expression. Scale bar, 20 μm. (E) Representative images of IHC staining for EMAP II expression (red) and Galectin-3 (green). Purple indicates co-expression. Scale bar, 100 μm. Note that compared to normoxia, lungs exposed to hyperoxia and harvested on Day 15 were severely dysplastic so that both bronchial epithelium and alveoli could not be imaged in the same field, although the same magnification as other images was used. (F) EMAP II concentration in tracheal aspirates by immunoblotting and quantification. Main effect of day, p=0.0187, interaction of oxygen: Day, p=0.711, n=3 per day). Data are represented as mean±1 s.e.m.

FIG. 2. EMAP II protein mediates macrophage chemoattraction in vivo. (A-E) Mice treated with either EMAP II or vehicle (injection) from days 3-15. (A) Schematic of EMAP II treatment in neonatal mice. (B) Representative immunohistochemical images of distal alveoli in lung sections of day 15 mice showing macrophage (Galectin-3, red) and (C) quantification by blinded analysis of Galectin-3 positive cells per high powered field (HPF) (n=4, p=0.00000235). (D,E) Immunoblot probed for IL1β in whole lung lysate in day 15 mice (normalized to β-actin, p=0.01, n=4). Scale bar, 100 μm.

Results are representative from four (B, C) or two independent experiments (D, E). Data are represented as mean±1 s.e.m.

FIG. 3. Lungs treated with EMAP II present BPD-like phenotype. The experimental design is the same as FIG. 2A. (A) Comparison of distal alveolar structure in inflation fixed lungs (25 mmHg) and sacrificed on day 15 by (B) MLI and (C) RAC by blinded observer analysis (n=8, p=0.03337). (D) Biophysical parameters of lung function compliance, resistance, elastance were assessed (n=3-6, p=0.011, 0.023, 0.008, respectively) and representative pulmonary flow loops presented. (E) Right ventricular hypertrophy quantified by Fulton's index (n=6 mice per group, p=0.00520) and (F) representative deposition of perivascular elastin (indicated by arrows) in distal lung tissue sections stained for Masson's Trichrome. Scale bars, (A) 100 μm, (F) 10 μm. Results are representative from three (A-C, F) or two (D-E) independent experiments. Data are represented as mean±1 s.e.m.

FIG. 4. Neutralizing EMAP II limits macrophage recruitment both in vitro and in vivo (A-D). (A) Quantification of Transwell-migrated macrophages in response to EMAP II vehicle (PBS), non-specific IgG, and EMAP II pre-incubated with varying concentrations of anti-EMAP II (n=2-4 replicates, p=0.0044, one-way ANOVA across treatments). Schematic of neonatal hyperoxia exposure protocol used to induce BPD, inj. (injection) of Anti-EMAP II or IgG. (C) Representative immunohistochemical images of distal alveoli in lung sections showing macrophages (Galectin-3, red) and (D) number of Galectin-3 positive cells per high power field (HPF), quantified by blinded analysis (n=4 mice, p=0.000457). Scale bars, 100 μm. Results are representative of samples collected from four (D, E) and two (A) independent experiments. Data are represented as mean±1 s.e.m.

FIG. 5. Rescued lung structure and function of BPD mice treated with anti-EMAP II. The experimental design is the same as FIG. 4c. (A) Comparison of distal alveolar structure in inflation fixed lungs (25 mmHg) sacrificed on day 15 by (B) MLI and (C) RAC by blinded observer analysis (n=8, p=0.0337, p=0.089). (D) Biophysical parameters of lung function compliance, resistance, elastance were assessed between hyperoxia groups (n=6-8 mice, p=0.00642, 0.000209, 0.00183) and representative pulmonary flow loops presented. (E) Right ventricular hypertrophy quantified by Fulton index (ratio of right ventricular (RV) weight to left ventricular (LV) plus septal (S) weight, n=3, p=0.00537) and (F) representative deposition of perivascular elastin (depicted in arrows) in distal lung tissue sections stained for Masson's Trichrome. Scale bars, (A) 100 μm, (E) 10 μm. Results are representative from four (A-F) or two (D-F) independent experiments. Data are represented as mean±1 s.e.m.

FIG. 6. Neutralizing EMAP II limited macrophage recruitment and reduced inflammation induced by high oxygen. (A,B) Representative immunoblot probed for IL1β in whole lung lysate in day 15 mice and quantified (n=3, normalized to β-actin, p=0.0498). (C) mRNA expression of inflammatory Il1b, Il6, Tnf, and chemokine genes Ccl2, Ccl9 in lungs determined by qPCR calculated on the basis of Hprt, Eef2, and Rpl13a expression (n=6-7, p=0.0195, 0.0489, 0.00594, 0.00227, 0.0889). Samples are from three independent experiments (A-C). Data are represented as mean±1 s.e.m.

FIG. 7. Perivascular EMAP II expression. Representative images of IHC co-staining for endomucin (green) and EMAP II (red) in Lungs of neonatal day 5 mice exposed to either normoxia or hyperoxia. Scale bar, 20 μm.

FIG. 8. EMAP II protein induced compensatory mechanisms. The experimental design is the same as FIG. 2A. (A) mRNA expression of Kdr and Flt1 in lung tissue was determined by qPCR, calculated on the basis of Eef2, and Rpl13a expression (n=4, p=0.130, 0.582). Values are expressed as arithmetic mean±1 s.e.m. (B) Comparison of body weight on day 15 of life (n=6, p=0.0258). (C) Biophysical parameters of lung tissue damping and tissue elastance were assessed (n=3-6 mice, p=0.00466, 0.00928). (D, E) Immunoblot of SFTPC protein expression and mRNA expression of Sftpc determined by qPCR calculated on the basis of Hprt, and Rpl13a expression (n=4, p=0.03, 0.0315). Data are represented as mean±1 s.e.m.

FIG. 9. Neutralizing EMAP II compensatory mechanisms in BPD mice. The experimental design is the same as FIG. 4C. (A) Representative images of antibody deposition (arrows) in day 15 lungs. Scale bars, 20 (B) Comparison of body weight on day 15 of life (n=17 per group, p=0.00789). (C) Quantification of immunofluorescent TUNEL assessment of apoptosis in day 15 lungs by blinded observer analysis (n=8, 3 fields per mouse, main effect of oxygen, p=0.0000236, main effect of antibody, p=0.728, interaction of antibody: oxygen, p=0.732). (D) Quantification of Surfactant protein C expression by Western blotting densitometry (n=6 per group, p=0.732, two-way ANOVA). (E) Biophysical parameters of lung tissue dampening and tissue elastance were assessed (n=3-6 mice, p=0.00508, 0.0103). Data are represented as mean±1 s.e.m.

### DETAILED DESCRIPTION

The present invention will now be described more fully hereinafter, in which preferred embodiments of the invention are described. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

The present inventors have found that the polypeptide EMAP II is highly associated with the development of BPD in infants and blocking the activity of EMAP II results in a reversal of the severe phenotype of BPD and suppression of pro-inflammatory and chemotactic genes within the lung.

The present disclosure provides pharmaceutical compositions comprising antagonists of Endothelial Monocyte-Activating Polypeptide (EMAP II) and methods of using antagonists of EMAP II for the treatment and prevention of lung disease, specifically BPD in a subject, specifically in infants. The disclosure further provides methods of treating and/or preventing BPD in infants undergoing supportive care with hyperoxia.

EMAP II (Aimp1) encodes one component of the Multi-Aminoacyl tRNA Synthetase Complex, is ubiquitously expressed, and is conserved across species. EMAP II is defined by its secreted, cleaved extracellular functions with recent studies focusing on its anti-angiogenic properties (21-25). EMAP II has also been indirectly shown to recruit macrophages in various injury models (26-28). EMAP II expression localizes between the epithelial/mesenchymal interface in early stages of normal murine lung development, while later saccular and alveolar developmental stages find low levels of EMAP II expression confined to the perivasculature (29, 30).

The present inventors have previously identified an association between elevated EMAP II levels and BPD in premature baboon and human infants (31). The inventors show here that EMAP II drives macrophage recruitment in BPD, which intensifies the inflammatory state. Using three mouse models, the inventors identified sources of EMAP II throughout BPD progression and showed functional roles for EMAP II in the disease progression of severe BPD. The inventors determined not only that its chemotactic role on macrophages leads to an inflammatory state exacerbating the development of BPD, but also represents a specific upstream, novel target for preventing BPD development.

### Pharmaceutical Compositions

In some embodiments, the present disclosure provides a pharmaceutical composition comprising (a) a therapeutically effective amount of an antagonist of endothelial monocyte-activating polypeptide II (EMAP II), and (b) a pharmaceutically suitable carrier.

An antagonist of EMAP II is a molecule, compound, protein, or ligand that blocks or reduces EMAP II-activity. An antagonist that inhibits EMAP II activity includes compounds that specifically bind to EMAP II (e.g., an antibody, more specifically a neutralizing antibody), compounds that downregulate EMAP II expression (e.g., an antisense oligonucleotide), EMAP II receptor antagonists or fragments of EMAP II receptor.

Such antagonists may be antibodies (including polyclonal and monoclonal antibodies, antibody fragments, humanized or chimeric antibodies, etc.) that retain the variable region that specifically binds to EMAP II. The antibodies may be of any type of immunoglobulin, including but not limited to IgG and IgM immunoglobulins. The antibodies may be of any suitable origin, such as chicken, goat, rabbit, horse, etc., but are preferably mammalian and most preferably human. The antibody may be administered directly or through an intermediate that expresses the antibody in the subject. Examples of antibodies to EMAP II are provided in U.S. Pat. No. 5,641,867 to Stern et al. Examples of the different forms of therapeutic antibodies are given in U.S. Pat. No. 5,622,700 to Jardieu et al., the disclosure of which is incorporated herein by reference in their entirety. Suitable antibodies include neutralizing antibodies to EMAP II. In the examples, a polyclonal antibody was used (which was disclosed in U.S. Pat. No. 7,537,757, which is incorporated by reference in its entirety). The antibody bind to antibody that binds to an epitope of Endothelial Monocyte Activating Polypeptide II (EMAP II), wherein the epitope consists of the amino acid sequence of SEQ ID NO:13 (Asp-Ala-Phe-Pro-Gly-Glu-Pro-Asp-Lys-Glu-Leu-Asn-Pro).

The antagonists of EMAP II also may include compounds that downregulate EMAP II expression. Suitable compounds include, for example, antisense oligonucleotides that bind to EMAP II mRNA and disrupt translation thereof, or oligonucleotides that bind to EMAP II DNA and disrupt transcription thereof. Such oligonucleotides may be natural or synthetic (such as described in U.S. Pat. No. 5,665,593 to Kole, the disclosure of which is incorporated by reference herein in its entirety), and are typically at least 4, 6 or 8 nucleotides in length, up to the full length of the corresponding DNA or mRNA. Such oligonucleotides are selected to bind to the DNA or mRNA by Watson-Crick pairing based on the known sequence of the EMAP II DNA as described in U.S. Pat. No. 5,641,867 to Stern et al., the contents of which are incorporated by reference in its entirety. For example, an antisense oligonucleotide of the invention may consist of a 4, 6 or 8 or more nucleotide oligonucleotide having a base sequence corresponding to the EMAP II DNA sequence disclosed in Stern et al, supra, up to 20, 30, or 40 nucleotides in length, or even the full length of the DNA sequence. In addition, such compounds may be identified in accordance with known techniques as described in WO 01/52879, which is incorporated by reference in its entirely.

Antagonists that are nucleotides or proteins (e.g., antibodies) may be administered either directly or through a vector intermediate that expresses the same in the subject. Thus vectors used to carry out the present invention are, in general, RNA virus or DNA virus vectors, such as lentivirus vectors, papovavirus vectors (e.g., SV40 vectors and polyoma vectors), adenovirus vectors and adeno-associated virus vectors. See generally T. Friedmann, Science 244, 1275 16 (June 1989).

Examples of lentivirus vectors that may be used to carry out the present invention include Moloney Murine Leukemia Virus vectors, such as those described in U.S. Pat. No. 5,707,865 to Kohn. Any adenovirus vector can be used to carry out the present invention. See, e.g., U.S. Pat. Nos. 5,518,913, 5,670,488, 5,589,377; 5,616,326; 5,436,146; and 5,585,362. The adenovirus can be modified to alter or broaden the natural tropism thereof, as described in S. Woo, Adenovirus redirected, Nature Biotechnology 14, 1538 (November 1996). Any adeno-associated virus vector (AAV vector) can also be used to carry out the present invention. See, e.g., U.S. Pat. Nos. 5,681,731; 5,677,158; 5,658,776; 5,658,776; 5,622,856; 5,604,090; 5,589,377; 5,587,308; 5,474,935; 5,436,146; 5,354,678; 5,252,479; 5,173,414; 5,139,941; and 4,797,368.

The regulatory sequences, or the transcriptional and translational control sequences, in the vectors can be of any suitable source, so long as they effect expression of the heterologous nucleic acid encoding the desired antagonist in the target cells. For example, commonly used promoters are the LacZ promoter, and promoters derived from polyoma, Adenovirus 2, and Simian virus 40 (SV40). See, e.g., U.S. Pat. No. 4,599,308. The heterologous nucleic acid may encode any product that inhibits the expression of the EMAP II gene in cells infected by the vector, such as an antisense oligonucleotide that specifically binds to the EMAP II mRNA to disrupt or inhibit translation thereof, a ribozyme that specifically binds to the EMAP II mRNA to disrupt or inhibit translation thereof, or a triplex nucleic acid that specifically binds to the EMAP II duplex DNA and disrupts or inhibits transcription thereof.

All of these may be carried out in accordance with known techniques, as (for example) described in U.S. Pat. Nos. 5,650,316; 5,176,996; and 5,650,316 for triplex compounds, in U.S. Pat. Nos. 5,811,537; 5,801,154; and 5,734,039 for antisense compounds, and in U.S. Pat. Nos. 5,817,635; 5,811,300; 5,773,260; 5,766,942; 5,747,335; and 5,646,020 for ribozymes (the disclosures of which are incorporated by reference herein in their entirety). The length of the heterologous nucleic acid is not critical so long as the intended function is achieved, but the heterologous nucleic acid is typically from 5, 8, 10 or 20 nucleic acids in length up to 20, 30, 40 or 50 nucleic acids in length, up to a length equal the full length of the EMAP II gene.

Once prepared, the recombinant vector can be reproduced by (a) propagating the vector in a cell culture, the cell culture comprising cells that permit the growth and reproduction of the vector therein; and then (b) collecting the recombinant vector from the cell culture, all in accordance with known techniques. The viral vectors collected from the culture may be separated from the culture medium in accordance with known techniques, and combined with a suitable pharmaceutical carrier for administration to a subject. Such pharmaceutical carriers include, but are not limited to, sterile pyrogen-free water or sterile pyrogen-free saline solution. If desired, the vectors may be packaged in liposomes for administration, in accordance with known techniques.

The dosage of the recombinant vector administered will depend upon factors such as the particular disorder, the particular vector chosen, the composition of the vector, the condition of the patient, the route of administration, etc., and can be optimized for specific situations. In general, the dosage is from about 10⁷, 10⁸, or 10⁹ to about 10¹¹, 10¹², or 10¹³ plaque forming units (pfu).

The term “pharmaceutically acceptable” as used herein means that the carrier is suitable for administration to a subject to achieve the treatments described herein, is compatible with any other ingredients in the composition, and is not unduly deleterious to the patient in light of the severity of the disease and necessity of the treatment.

By “pharmaceutically acceptable carrier” we mean any and all solvents, dispersion media, coatings, antibacterial and antifungal agents, isotonic and absorption delaying agents, and the like that are physiologically compatible. In one embodiment, the carrier may be suitable for inhalation administration (e.g. aerosol). Alternatively, the carrier can be suitable for intravenous, parenteral, intraperitoneal, intramuscular, sublingual or oral administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the antagonist, use thereof in the pharmaceutical compositions of the invention is contemplated. Additional agents or therapies can also be incorporated into the compositions.

The pharmaceutical compositions described herein may be formulated with the antagonists of EMAP II in a pharmaceutical carrier in accordance with known techniques. See, e.g., Remington, The Science And Practice of Pharmacy 9th Ed. (A. R. Gennaro, ed., Mack Publishing Co., Easton, Pa., 1995).

The compositions of the invention include those suitable for oral, rectal, buccal (e.g., sub-lingual), parenteral (e.g., subcutaneous, intraperitoneal, intramuscular, intradermal, intraarticular, intrathecal, intralesion or intravenous), topical (i.e., both skin and mucosal surfaces, including airway surfaces), inhalation and transdermal administration. In some embodiments, the compositions are prepared for inhalation (aerosol) administration. The most suitable route in any given case will depend on the nature and severity of the condition being treated and on the characteristics of the particular antagonist which is being used. In the practice of the present invention, preferred routes of administration include intravenous, intraperitoneal, and inhalation administration.

The pharmaceutical compositions may optionally include one or more additional ingredients depending on the mode of administration and the characteristics of the antagonist to maintain the activity of the antagonist during storage and preparation. Suitably, in some embodiments, the pharmaceutical composition may contain additives such as pH-adjusting additives, anti-microbial preservatives, stabilizers and the like. In particular, useful pH-adjusting agents include, but are not limited to, for example, acids, such as hydrochloric acid, bases or buffers, such as sodium lactate, sodium acetate, sodium phosphate, sodium citrate, sodium borate, or sodium gluconate. Useful microbial preservatives are known in the art and include, but are not limited to, methylparaben, propylparaben, and benzyl alcohol. The microbial preservative is typically employed when the composition is placed in a vial designed for multidose use.

Therapeutic compositions typically must be sterile and stable under the conditions of manufacture and storage. The composition can be formulated as a solution, microemulsion, liposome, membrane nanoparticle or other ordered structure suitable to the proposed drug concentration. The carrier can be a solvent or dispersion medium containing, for example, water, saline, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, such as, monostearate salts and gelatin.

Sterile injectable solutions can be prepared by incorporating the antagonist (e.g. EMAP II antibody) in the required amount in an appropriate solvent with one or a combination of ingredients, as required, followed by filtered sterilization. Generally, dispersions are prepared by incorporating the antagonist into a sterile vehicle which contains a basic dispersion medium and other ingredients. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. The EMAP II antagonist of the present invention also may be formulated with one or more additional compounds that enhance the solubility of the EMAP II antagonist.

The pharmaceutical compositions may include one or more additional agents or therapies that can treat the lung condition, including BPD. In some embodiments the pharmaceutical composition is formulated to comprise or consist essentially of the antagonist of EMAP II and at least one additional agent or therapy.

In one embodiment of the invention, the antagonists or pharmaceutical compositions of the invention are administered directly to the lungs of the subject by any suitable means, but are preferably administered by administering an aerosol suspension of respirable particles comprised of the antagonist, which the subject inhales. The antagonist can be aerosolized in a variety of forms, such as, but not limited to, dry powder inhalants, metered dose inhalants, or liquid/liquid suspensions. The respirable particles may be liquid or solid.

Solid or liquid particulate forms of the antagonist prepared for practicing the present invention should include particles of respirable size: that is, particles of a size sufficiently small to pass through the mouth and larynx upon inhalation and into the bronchi and alveoli of the lungs of an infant. In general, particles ranging from about 1 to 10 microns in size are within the respirable range. In some embodiments, the particle size is extra-fine particle delivery, for example, less than 2.5 microns. Not to be bound by any theory, but smaller particle size may lead to increased efficacy of delivery of the composition. Particles of non-respirable size which are included in the aerosol tend to be deposited in the throat and swallowed, and the quantity of non-respirable particles in the aerosol is preferably minimized. The particulate pharmaceutical composition may optionally be combined with a carrier to aid in dispersion or transport. A suitable carrier such as a sugar (i.e., lactose, sucrose, trehalose, mannitol) may be blended with the antagonist(s) in any suitable ratio (e.g., a 1 to 1 ratio by weight).

Suitably, the compositions may be formulated into aerosols to be administered by inhalation. Aerosols of liquid particles comprising the antagonist may be produced by any suitable means, such as with a pressure-driven aerosol nebulizer or an ultrasonic nebulizer. See, e.g., U.S. Pat. No. 4,501,729, incorporated by reference in its entirety.

Nebulizers are commercially available devices known in the art which transform solutions or suspensions of the active ingredient into a therapeutic aerosol mist either by means of acceleration of compressed gas, typically air or oxygen, through a narrow orifice or by means of ultrasonic agitation. Several types of nebulizers are available, including, for example, jet nebulizers, ultrasonic nebulizers, vibrating mesh nebulizers. Jet nebulizers are driven by compressed air. Suitable compositions for use in nebulizers consist of the active ingredient in a liquid carrier, the active ingredient comprising up to 40% w/w of the composition, but in some embodiments, preferably less than 20% w/w. In some embodiments, the carrier is water (and most preferably sterile, pyrogen-free water) or a dilute aqueous alcoholic solution, preferably made isotonic but may be hypertonic to body fluids by the addition of, for example, sodium chloride. Optional additives include preservatives if the composition is not made sterile, for example, methyl hydroxybenzoate, antioxidants, volatile oils, buffering agents and surfactants.

Aerosols of solid particles comprising the antagonist may likewise be produced with any solid particulate medication aerosol generator. Aerosol generators for administering solid particulate medicaments to a subject are known in the art, for example, generate a volume of aerosol containing a predetermined metered dose of a medicament at a rate suitable for human administration. For example, a solid particulate aerosol generator may be, but not limited to, an insufflator or a metered dose inhaler. Suitable compositions for administration by insufflation include finely comminuted powders which may be delivered by means of an insufflator or taken into the nasal cavity in the manner of a snuff. Dry powder inhalers are devices used to deliver drugs, especially proteins to the lungs. Some of the commercially available dry powder inhalers include Spinhaler (Fisons Pharmaceuticals, Rochester, N.Y.) and Rotahaler (GSK, RTP, NC).

The powder employed in the insufflator may consist either solely of the active ingredient or of a powder blend comprising the active ingredient, a suitable powder diluent, such as lactose, and an optional surfactant. The antagonist typically comprises from 0.1 to 100 w/w of the composition. Metered dose inhalers are pressurized aerosol dispensers, typically containing a suspension or solution composition of the antagonist in a liquefied propellant. During use these devices discharge the composition through a valve adapted to deliver a metered volume, typically from 10 to 200 μl, to produce a fine particle spray containing the antagonist. Suitable propellants include certain chlorofluorocarbon compounds, for example, dichlorodifluoromethane, trichlorofluoromethane, dichlorotetrafluoroethane and mixtures thereof. The composition may additionally contain one or more co-solvents, for example, ethanol, surfactants, such as oleic acid or sorbitan trioleate, antioxidants and suitable flavoring agents.

Any propellant may be used in carrying out the present invention, including both chlorofluorocarbon-containing propellants and non-chlorofluorocarbon-containing propellants. Thus, fluorocarbon aerosol propellants that may be employed in carrying out the present invention including fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to: CF3-CHF—CF2H; CF3-CH2-CF2H; CF3-CHF—CF3; CF3-CH2-CF3; CF3-CHC1-CF2C1; CF3-CHC1-CF3; cy-C(CF2)3-CHC1; CF3-CHC1-CH2C1; CF3-CHF—CF2C1; CF3-CHC1-CFHC1; CF3-CFC1-CFHC1; CF3-CF2-CF2H; CF3-CF2-CH3; CF2H—CF2-CFH2; CF3-CF2-CFH2; CF3-CF2-CH2C1; CF2H—CF2-CH3; CF2H—CF2-CH2C1; CF3-CF2-CF2-CH3; CF3-CF2-CF2-CF2H; CF3-CHF—CHF—CF3; CF3-O—CF3; CF3-O—CF2H; CF2H—H—O—CF2H; CF2H—O—CFH2; CF3-O—CH3; CF3-O—CF2-CF2H; CF3-O—CF2-O—CF3; cy-CF2-CF2-O—CF2-; cy-CHF—CF2-O—CF2-; cy-CH2-CF2-O—CF2-; cy-CF2-O—CF2-O—CF2-; CF3-O—CF2-Br; CF2H—O—CF2-Br; and mixtures thereof, where “cy” denotes a cyclic compound in which the end terminal covalent bonds of the structures shown are the same so that the end terminal groups are covalently bonded together. Particularly preferred are hydrofluoroalkanes such as 1,1,1,2-tetrafluoroethane and heptafluoropropane. A stabilizer such as a fluoropolymer may optionally be included in compositions of fluorocarbon propellants, such as described in U.S. Pat. No. 5,376,359 to Johnson.

Methods of making compositions containing respirable dry particles of micronized antagonist of the present invention are known in the art. The aerosol, whether formed from solid or liquid particles, may be produced by the aerosol generator at a rate of about 10 to 150 liters per minute. Aerosols containing greater amounts of medicament may be administered more rapidly. Typically, each aerosol may be delivered to the patient for a period from about 30 seconds to about 20 minutes, with a delivery period of about five to ten minutes being preferred. Toxicity concerns at the higher level may restrict intravenous dosages to a lower level such as up to about 10 mg/kg. A dosage from about 10 mg/kg to about 50 mg/kg may be employed for oral administration. Typically, a dosage from about 0.5 mg/kg to 5 mg/kg may be employed for intramuscular injection. Preferred dosages are 1 μmol/kg to 50 μmol/kg, and more preferably 22 μmol/kg and 33 μmol/kg of the compound for intravenous or oral administration.

Regardless of the route of administration of the antagonists or compositions of the invention, the therapeutically effective dosage of any one active antagonist, the use of which is in the scope of present invention, will vary somewhat from antagonist to antagonist, and patient to patient, and will depend upon factors such as the age, weight and condition of the patient, and the route of delivery. Such dosages can be determined in accordance with routine pharmacological procedures known to those skilled in the art. For example, as a general proposition, a dosage from about 0.1 to about 50 mg/kg will have therapeutic efficacy, with all weights being calculated based upon the weight of the antagonist. Toxicity concerns at the higher level may restrict intravenous dosages to a lower level such as up to about 10 mg/kg. A dosage from about 10 mg/kg to about 50 mg/kg may be employed for oral administration. Typically, a dosage from about 0.5 mg/kg to 5 mg/kg may be employed for intramuscular injection. Preferred dosages are 1 μmol/kg to 50 μmol/kg, and more preferably 22 μmol/kg and 33 μmol/kg of the compound for intravenous or oral administration.

The doses of the compositions or antagonists may be provided as one or several prepackaged units.

The duration of the treatment is usually once or twice per day for a period of time that will vary by subject, but will generally last until the condition is essentially controlled. In some embodiments, the duration of treatment may be multiple times per day, twice a day, or once a day, and in some instances may be every other day or once a week depending on the state of the condition.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intra-arterial, intramuscular, or intrasternal injection and intravenous, intra-arterial, or kidney dialytic infusion techniques.

Compositions suitable for parenteral injection comprise the antagonist of EMAP II of the invention combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols (e.g., propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such compositions can be prepared, packaged, or sold in a form suitable for bolus administration or for continuous administration. Injectable compositions can be prepared, packaged, or sold in unit dosage form, such as in ampules, in multi-dose containers containing a preservative, or in single-use devices for auto-injection or injection by a medical practitioner. Such compositions can further comprise one or more additional ingredients including suspending, stabilizing, or dispersing agents.

The pharmaceutical compositions can be prepared, packaged, or sold in the form of a sterile injectable aqueous or oily suspension or solution. This suspension or solution can be formulated according to the known art. Such sterile injectable compositions can be prepared using a non-toxic parenterally-acceptable diluent or solvent, such as water or 1, 3-butanediol, for example. Other acceptable diluents and solvents include Ringer's solution, isotonic sodium chloride solution, and fixed oils such as synthetic mono- or di-glycerides. Other parenterally-administrable compositions which are useful include those which comprise the active ingredient in microcrystalline form, in a liposomal preparation, or as a component of a biodegradable polymer systems. Compositions for sustained release or implantation can comprise pharmaceutically acceptable polymeric or hydrophobic materials such as an emulsion, an ion exchange resin, a sparingly soluble polymer, or a sparingly soluble salt.

Compositions suitable for oral administration may be presented in discrete units, such as capsules, cachets, lozenges, or tablets, each containing a predetermined amount of the antagonist; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the antagonist and a suitable carrier (which may contain one or more additional ingredients).

In general, the compositions of the invention are prepared by uniformly and intimately admixing the antagonist with a liquid or finely divided solid carrier, or both, and then, if necessary, shaping the resulting mixture. For example, a tablet may be prepared by compressing or molding a powder or granules containing the antagonist, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder. Compositions of the present invention suitable for parenteral administration comprise sterile aqueous and non-aqueous injection solutions of the antagonist, which preparations are preferably isotonic with the blood of the intended recipient. These preparations may contain anti-oxidants, buffers, bacteriostats and solutes which render the composition isotonic with the blood of the intended recipient. Aqueous and non-aqueous sterile suspensions may include suspending agents and thickening agents.

The compositions may be presented in uni-dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an antagonist in a unit dosage form in a sealed container. The compound is provided in the form of a lyophilizate which is capable of being reconstituted with a suitable pharmaceutically acceptable carrier to form a liquid composition suitable for injection thereof into a subject.

The unit dosage form typically comprises from about 10 mg to about 10 grams of the compound. When the compound is substantially water-insoluble, a sufficient amount of emulsifying agent which is physiologically acceptable may be employed in sufficient quantity to emulsify the compound or salt in an aqueous carrier. Useful emulsifying agents include but are not limited to phosphatidyl choline and lecithin.

Solid dosage forms for oral administration include capsules, tablets, powders, and granules. In such solid dosage forms, the antagonist is admixed with at least one inert customary excipient (or carrier) such as, for example, but not limited to, sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent.

Tablets may be manufactured with pharmaceutically acceptable excipients such as inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or coated using known methods to achieve delayed disintegration in the gastrointestinal tract of a human, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the antagonist or compounds in a delayed manner. Examples of embedding compositions that can be used are polymeric substances and waxes. The antagonists can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols, and the like. Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin. Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

Compositions suitable for transdermal administration may be presented as discrete patches adapted to remain in intimate contact with the epidermis of the recipient for a prolonged period of time. Compositions suitable for transdermal administration may also be delivered by iontophoresis (see, for example, Pharmaceutical Research 3, 318 (1986)) and typically take the form of an optionally buffered aqueous solution of the antagonist. Suitable compositions comprise citrate or bis\tris buffer (pH 6) or ethanol/water and contain from 0.1 to 0.2M active ingredient.

Compositions suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof. Compositions suitable for buccal (sub-lingual) administration include lozenges comprising the antagonist in a flavored base, usually sucrose.

Optionally, the present invention provides liposomal compositions of the compounds disclosed herein and salts thereof. The technology for forming liposomal suspensions is well known in the art. When the antagonist is aqueous-soluble, using conventional liposome technology the same may be incorporated into lipid vesicles. In such an instance, due to the water solubility of the compound, the compound will be substantially entrained within the hydrophilic center or core of the liposomes. The lipid layer employed may be of any conventional composition and may either contain cholesterol or may be cholesterol-free. When the antagonist is water-insoluble, again employing conventional liposome formation technology, the compound may be substantially entrained within the hydrophobic lipid bilayer which forms the structure of the liposome. In either instance, the liposomes which are produced may be reduced in size, as through the use of standard sonication and homogenization techniques. Of course, the liposomal compositions containing the antagonists disclosed herein may be lyophilized to produce a lyophilizate which may be reconstituted with a pharmaceutically acceptable carrier, such as water, to regenerate a liposomal suspension.

The amount of antagonist of EMAP II in the composition may vary according to factors such as the disease state, age, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of antagonist calculated to produce the desired therapeutic effect in association with the required pharmaceutical carrier. The specification for the dosage unit forms of the invention are dictated by and directly dependent on (a) the unique characteristics of the antagonist and the particular therapeutic effect to be achieved, and (b) the limitations inherent in the art of compounding such as antagonist for the treatment of sensitivity in individuals.

### Methods of Treatment

In some embodiments the present disclosure provides a method of treating a lung condition in an infant in need thereof. The method comprises administering to the subject a pharmacologically effective amount of the pharmaceutical composition comprising at least one antagonist of EMAP II as described above.

By lung condition we mean a condition in which the lung of the subject (e.g. infant) has a chronic or acute lung disease, for example, bronchopulmonary dysplasia (BPD). BPD is a chronic lung disorder of infants and children. BPD is commonly found in infants with low birth weight and those who receive prolonged mechanical ventilation to treat respiratory distress syndrome (RDS). In its most severe form, BPD can result in secondary cardiovascular sequelae such as pulmonary hypertension (PH) that persist into adulthood and abnormal ventilatory response. The National Institute of Health has provided criteria for BPD into mild, moderate or severe (See Jobe, A H; Bancalari, E (June 2001). “Bronchopulmonary dysplasia”. Am J Respir Crit Care Med. 163 (7): 1726). The compositions of the present disclosure are contemplated to treat mild, moderate and severe forms of BPD. In some embodiments, the lung condition may be secondary pulmonary hypertension resulting from BPD.

By “therapeutically effective amount” we mean an amount effective, at dosages and for periods of time necessary, to achieve the desired therapeutic result, such as reduction, amelioration, inhibition of the lung condition, a reduction, inhibition, amelioration or one or more symptoms of the lung condition or of bronchopulmonary dysplasia, or a reduction of macrophage infiltration or pro-inflammatory markers into the lung of the subject.

Symptoms of BPD include, but are not limited to, symptoms of respiratory distress syndrome (RDS) including shortness of breath, rapid, shallow breathing, sharp pulling of the chest below and between the ribs with each breath, grunting sounds when breathing, flaring of the nostrils, arrested alveolar development, right ventricular hypertrophy, macrophage recruitment to the lungs, and heightened inflammatory state of the lungs (e.g. increase in inflammatory markers in the lung), impaired biophysical properties including insufficient oxygen exchange and inflammation, alveolar dysplasia, hypoplasia, loss of alveolar capillaries, hypoxia, respiratory failure, and mild fibrosis.

By “subject” we mean mammals and non-mammals. “Mammals” means any member of the class Mammalia including, but not limited to, humans, non-human primates such as chimpanzees and other apes and monkey species, mice, rats, dogs, cats livestock and horses. The term “subject” does not denote a particular age or sex. In some embodiments, the preferred subject is a human, and preferably a human infant. By “infant” we mean a young child between the ages of 0 days old to about 2 year old. In some embodiments, the infant is between 0 days and 1 year old. In some embodiments, the infant is a neonate. The term “neonate” or “newborn” refers to an infant in the first 28 days after birth, and applies to premature infants, postmature infants and full term infants. In some embodiments, premature infants are treated. In some embodiments, premature infants of low body weight are treated by the methods described herein.

By “treating” or “treatment” we mean the management and care of a subject for the purpose of combating and reducing the disease, condition, or disorder. The terms embrace preventative, i.e., prophylactic, and palliative treatments. Treating includes the administration of an antagonist of the present invention to prevent, ameliorate and/or improve the onset of the symptoms or complications, alleviating the symptoms or complications of the lung condition or disease, or eliminating the disease, condition, or disorder. Treating of a lung condition, including bronchopulmonary dysplasia, includes, but is not limited to, reducing, inhibiting, or preventing one or more symptom of the lung condition or BPD, delay in the progression of BPD. In some embodiments, the term treating an infant at risk of developing BPD includes preventing, inhibiting or reducing the severity of at least one symptom of BPD, and may include elimination of the development of BPD in the infant. As used herein, the term “treatment” is not necessarily meant to imply cure or complete abolition of BPD. Treatment may refer to the inhibiting or slowing of the progression of BPD, reducing the incidence of BPD, or preventing BPD. Alternatively stated, the present methods slow, delay, control, or decrease the likelihood or probability of BPD in the infant as compared to that which would occur in the absence of treatment.

By “ameliorate”, “amelioration”, “improvement” or the like we mean a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with the pharmaceutical compositions of the present invention, where the untreated subjects have, or are subject to developing, the same or similar disease, condition, symptom or the like. Amelioration of a disease, condition, symptom or assay parameter may be determined subjectively or objectively, e.g., by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of cell migration (e.g. macrophages) within a subject. Amelioration may be transient, prolonged or permanent or it may be variable at relevant times during or after the pharmaceutical compositions are administered to a subject or is used in an assay or other method described herein or a cited reference, e.g., within about 1 hour of the administration or use of the compositions of the present invention to about 3, 6, 9 months or more after a subject(s) has received the compositions of the present invention.

By “administering” we mean any means for introducing the pharmaceutical composition or antagonist of EMAP II of the present invention into the body. Examples include but are not limited to oral, buccal, sublingual, pulmonary, transdermal, transmucosal, and aerosol, as well as subcutaneous, intraperitoneal, intravenous, and intramuscular injection. Suitable forms of the pharmaceutical composition for different routes of administration are described more above.

A preferred method of administering the pharmaceutical compositions of the present invention for treatment of lung conditions, particularly bronchopulmonary dysplasia, is by inhalation (e.g. aerosol). Another suitable method of administration is oral or parenteral (e.g. intravenous).

In some embodiments, the subject is treated every day, in alternative embodiments, the subject is treated every other day, in further alternative embodiments, the subject is treated every third day after birth and/or after start of treatment.

In some embodiments, the method of the present disclosure further include administering at least one additional agent or therapy. Suitable additional agents or therapies are known by one skilled in the art and include, but are not limited to, a surfactant, oxygen therapy, ventilator therapy, a steroid, inhaled nitric oxide, diuretics, bronchodilators, fluid restriction, good nutrition, anti-inflammatory agents, CPAP, Vitamin A, caffeine, pulmonary hypertension therapy (sildenafil, Nitric Oxide, Milrinone, Epoprostenol), Azithromycin, Methylxanthines, and anti-viral.

In some embodiments, the present disclosure provides methods of treating an infant at risk of developing bronchopulmonary dysplasia (BPD) comprising administering a therapeutically effective amount of the pharmaceutical composition comprising an antagonist of EMAP II. In some embodiments, the treatment further includes an additional agent or therapy.

In some embodiments, the present invention provides a method of reducing the inflammation associated with BPD in a subject comprising administering to the subject a therapeutically effective amount of the pharmaceutical composition comprising an antagonist of EMAP II. In some embodiments, the treatment results in the decrease of inflammatory markers within the lung of the subject. Specifically, in some embodiments, the method results in the suppression of the expression of pro-inflammatory genes Tnfa, Il6, Il1b and chemotactic genes Ccl2, Cc19. This reduction in the pro-inflammatory genes and chemotactic genes occurs with the reversal of the severe BPD phenotype.

Other embodiments provide methods of reducing macrophage infiltration into the lungs of a subject suffering from bronchopulmonary dysplasia. The method comprises administering a therapeutically effective amount of the pharmaceutical composition of the present invention. The administration results in a reduction of the macrophage infiltrating the lung of the subject, resulting in a reduction of the pro-inflammatory inflammation associated with the infiltration of macrophages. Not to be bound by any theory, but the administration of an antagonist to EMAP II is believed to suppress EMAP II chemoattractant abilities to recruit macrophages and also to inhibit EMAP II inflammatory properties to alleviate pulmonary biophysical abnormalities associated with hyperoxia induced BPD, including decreased resistance, decreased tissue damping, and decreased airway space. The reduction in the ability to chemoattract macrophages and reduction in the inflammatory response in lungs may lead to the prevention BPD and secondary pulmonary hypertension related to BPD. In some embodiments, the reduction in macrophage recruitment leads to a reduction of inflammatory mediators secreted by macrophages, for example, IL1β or TNF-α.

This disclosure also provides kits. The kits can be suitable for use in the methods described herein. Suitable kits include a kit for treating lung condition, including BPD comprising a pharmaceutical composition comprising at least one antagonist of EMAP II. In one aspect, the kit provides pharmaceutical composition comprising an antagonist of EMAP II in amounts effective for treating BPD. In some aspects, instructions on how to administer the pharmaceutical composition and/or active agents are provided.

The following non-limiting examples are included for purposes of illustration only, and are not intended to limit the scope of the range of techniques and protocols in which the compositions and methods of the present invention may find utility, as will be appreciated by one of skill in the art and can be readily implemented.

### EXAMPLES

### Example 1: EMAP II Mediates Macrophage Migration in the Development of Hyperoxia-Induced Lung Disease of Prematurity

Rationale:

Myeloid cells are key factors in the progression of BPD pathogenesis. Endothelial Monocyte-Activating Polypeptide II (EMAP II) mediates myeloid cell trafficking. In BPD, the origin and physiological mechanism by which EMAP II affects pathogenesis in BPD is unknown.

Objective:

To determine the functional consequences of elevated EMAP II levels in the pathogenesis of murine BPD and investigate EMAP II neutralization as a therapeutic strategy.

Methods:

Three neonatal mice models were used: (1) BPD (hyperoxia), (2) EMAP II delivery, (3) BPD with neutralizing EMAP II antibody treatments. Chemokinic function of EMAP II and its neutralization were assessed by migration in vitro and in vivo. The inventors determined the location of EMAP II by immunohistochemistry, pulmonary pro-inflammatory and chemotactic gene expression by quantitative PCR and immunoblotting, lung outcome by pulmonary function testing and histological analysis, and right ventricular hypertrophy assessed by Fulton's Ind7ex.

Measurements and Main Results:

In BPD, EMAP II initially is a bronchial club-cell-specific-protein derived factor that later is expressed in GAL-3⁺ macrophages as BPD progresses. Continuous elevated expression corroborates with baboon and human BPD. Prolonged elevation of EMAP II levels recruit GAL-3⁺ macrophages followed by an inflammatory state that resembles a severe BPD phenotype characterized by decreased pulmonary compliance, arrested alveolar development, and signs of pulmonary hypertension. In vivo pharmacological EMAP II inhibition suppressed pro-inflammatory genes Tnfa, Il6, Il1b and chemotactic genes, Ccl2, Ccl9 and reversed the severe BPD phenotype.

Conclusions:

EMAP II is sufficient to induce macrophage recruitment, worsens BPD progression, and represents a targetable mechanism of BPD development.

### Abbreviations

Anti-EMAP II: EMAP II neutralizing antibody BPD: Bronchopulmonary Dysplasia

EMAP II: Endothelial Monocyte-Activating Polypeptide II MLI: Mean Linear Intercept

PH: Pulmonary Hypertension RAC: Radial Alveolar Count

Methods Mice Studies

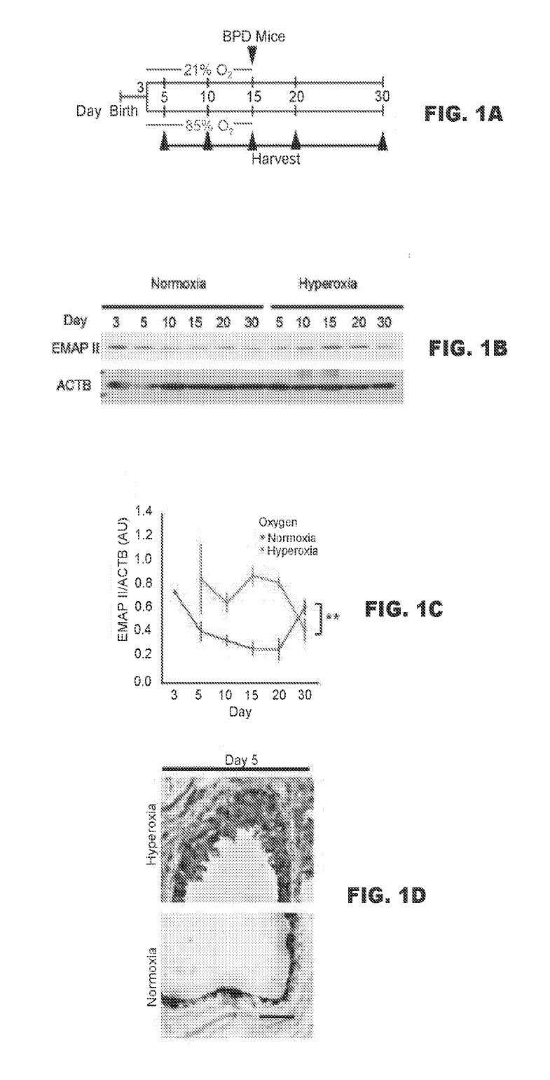

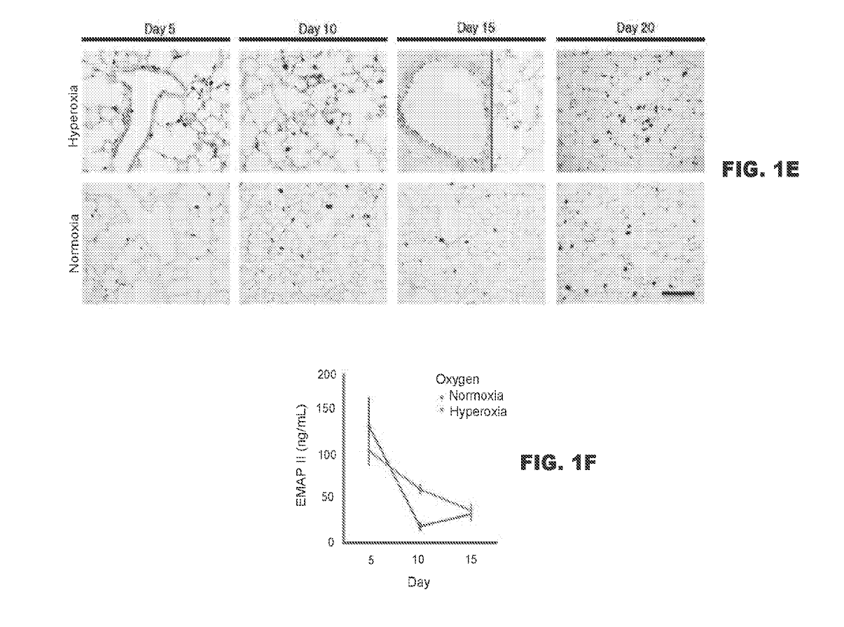

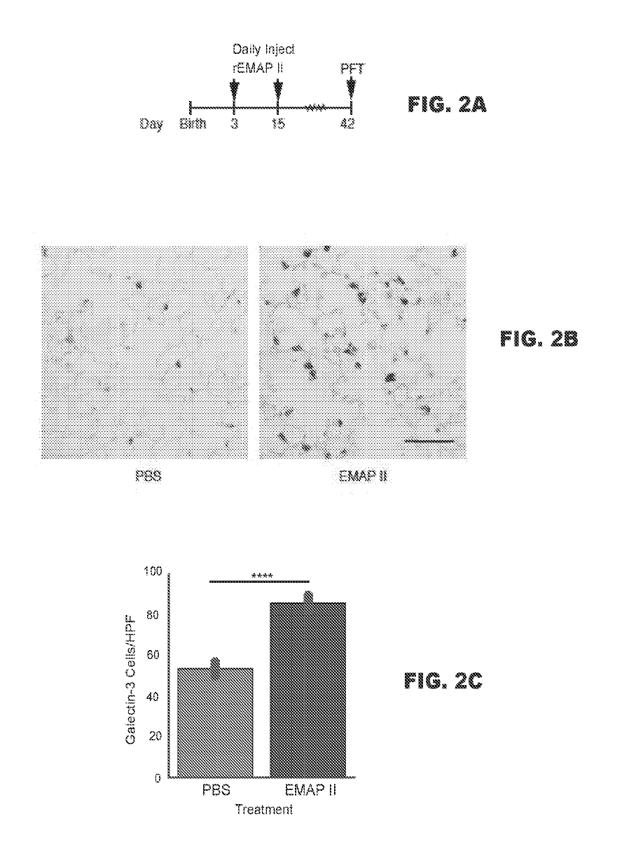

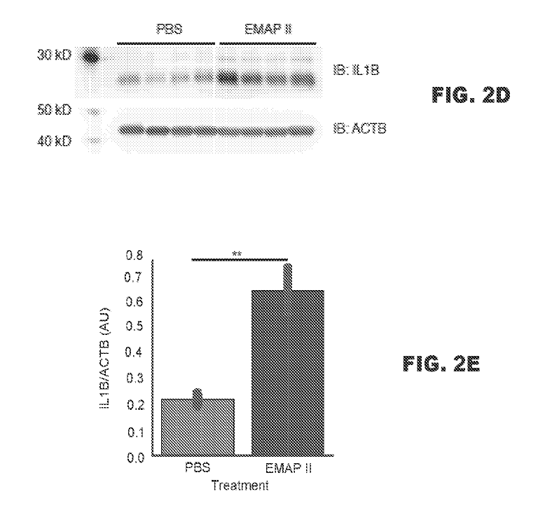

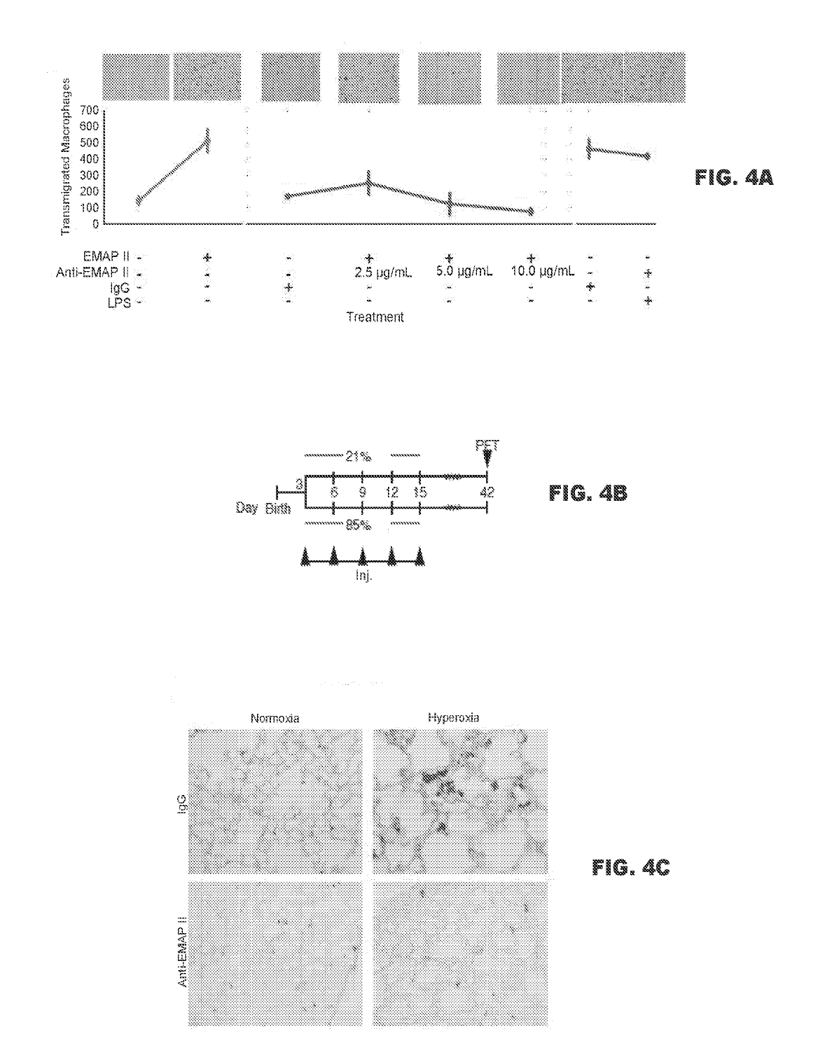

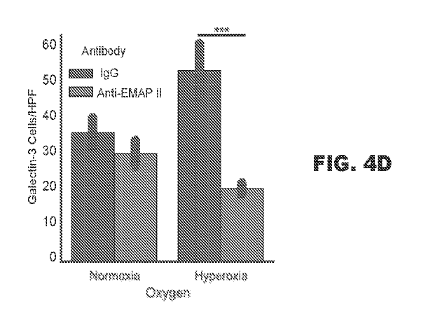

C57BL/6 mice were obtained from Jackson Laboratories. Studies complied with the animal protocols approved by the Indiana University Institutional Animal Care and Use Committee. Newborn pups were randomly selected for treatment groups while mice dams were exchanged every 24 hours to prevent oxygen toxicity. For details on normoxia and hyperoxia treatments, see FIG. 1A. Regarding recombinant EMAP II injection studies, see FIG. 2A. Antibodies neutralizing EMAP II were delivered according to FIG. 4B. For further details, see Supplementary Methods.

Quantitative PCR and Immunoblotting

RNA extraction, data collection, and analysis were performed according to the methods in previous study (32). See Supplementary Methods for further details on protein extraction and immunoblotting. Supplementary Table details antibodies and dilutions used in these studies.

Lung Microscopy and Morphometry Analysis

Lung tissue sections were prepared as previously described (33). Antigens on lung sections of five microns were retrieved and stained with antibodies according to Supplementary Table. Mean linear intercepts and radial alveolar counts were calculated from H&E stained sections. GAL-3⁺ counts were performed in blinded manner, decoded, and analyzed using Python 2.7. Further details are listed on Supplementary Methods.

Lung Functional Studies

Only male mice were tested for pulmonary functions to avoid possible hormonal issues. Mice were anesthetized with ketamine (100 mg/kg) and xylazine (6 mg/kg) followed by pancuronium (1 mg/kg) to induce paralysis. A metal cannulus was inserted through a small tracheal incision followed by single-model and complex model measurements of lung function using FlexiVent Software (SCIREQ Inc.).

Transwell Migration Study

RAW264.7 cells (ATCC) were cultured in phenol-red free DMEM media containing 10% FBS, antimicrobial and antifungal supplement, 5 mM HEPES, and 5 mM L-Glutamine until approximately 70-80% confluent. The media exchanged for transmigration media containing phenol-red free DMEM, 1% FBS for 2 hours before being scraped, incubated in CD16/32 to block non-specific F′ ab interactions on ice for approximately 15 minutes, washed, centrifugation at 400×g for 5 minutes at 4° C. and aspirated. 5×10⁴ cells were resuspended in transmigration media and loaded into a single 5.0-micron pore transwell insert. Inactivation of EMAP II protein by boiling for 30 minutes at 100° C. or pre-incubated with EMAP II neutralizing antibody at room temperature for 30 minutes at respective dosages. LPS (Serotype E. Coli 055:B4, Sigma) was also pre-incubated with EMAP II neutralizing antibody. The bottom inserts were filled with 500 microliters containing the listed treatments. Transmigration occurred for 4 hours at 37° C., fixed in 4% paraformaldehyde (w/v in PBS) overnight, and stained in crystal violet solution. Images were captured at 20× magnification on DP70 using MicroSuite Biological Software. n=4-6 for each treatment.

Results

EMAP II Levels in Lung Disease of Prematurity.

The inventors exposed neonatal mice to 85% 02 saturation level (i.e. hyperoxia) compared to room air (normoxia) during lung alveologenesis to induce BPD formation (FIG. 1A). EMAP II protein levels were quantified by immunoblotting (FIG. 1B). Confirming previous studies, EMAP II expression was perivascular in normoxic day 5 (FIG. 7). Compared to mice at normoxia, EMAP II levels were significantly elevated in lungs of mice exposed to hyperoxia over time, peaking at postnatal day 15 (FIG. 1C) (from hereon, mice exposed to hyperoxia and analyzed between 5 to 15 days are termed “early BPD mice,” mice analyzed at later time points are termed-15 days, “BPD mice,” and for 20 days and beyond, “late BPD”). However, analysis of EMAP II protein levels in tracheal aspirates of BPD mice revealed an early increase at day 10 but a decline toward that of normoxia control mice by day 15 (FIG. 1F).

EMAP II expression differs in location during BPD formation.

A significant increase over time in whole lung but decreasing trend in tracheal aspirates suggested that EMAP II expression is localized and compartmentalized in response to hyperoxia. EMAP II has been shown to augment inflammatory cell counts (34). We proposed that the localization of EMAP II would be distributed in cells near the tracheal aspirate collection site and thus histological analysis by co-staining EMAP II with Galectin-3 (known as GAL-3), an activation and differentiation marker of macrophages was performed. In contrast to normal perivascular localization of EMAP II expression, by day 5, EMAP II expression was found in both proximal bronchiolar epithelial-rich regions, indicated by club-cell-specific-protein expression, and perivasculature (FIG. 1D). By day 10, EMAP II expression was limited to GAL-3⁺ macrophages that were located in both bronchiole and distal airways (FIG. 1E); subsequently by day 15, EMAP II was localized only within macrophages of the distal airways. In agreement with the localization moving distally away from bronchiolar airways, analysis of tracheal aspirates showed a significant decrease in EMAP II expression.

In Vivo Effect of EMAP II on Macrophages.

As there was a recruitment of macrophages over time found in BPD mice (FIG. 1E), we postulated that excess EMAP II in early BPD directly recruited macrophages. We administered recombinant EMAP II to mice until the time point when there was maximal EMAP II expression on day 15 (FIG. 2A). The dosage followed previous studies that determined EMAP II's alternate moonlighting anti-angiogenic role (35); as previously observed, there were decreased angiogenic genes without a compensatory effect on transcription (FIG. 8A,B). We found a significant increase in the number of macrophages in lungs of mice administered EMAP II as compared to controls (FIG. 2B,C). This suggested that there was macrophage chemoattraction by EMAP II.

In BPD, particular focus has been attributed to the pro-inflammatory cytokine, interleukin-1 beta (IL-1β). As it is primarily secreted by macrophages, and having seen a significant increase in macrophage recruitment, we evaluated IL-1β expression in whole lung (FIG. 2D). There was significantly elevated IL-1β expression in lungs administered EMAP II (FIG. 2D, E) suggesting contribution to macrophage pulmonary sequestration.

Effect of EMAP II on Lung Structure and BPD Pulmonary Outcomes.

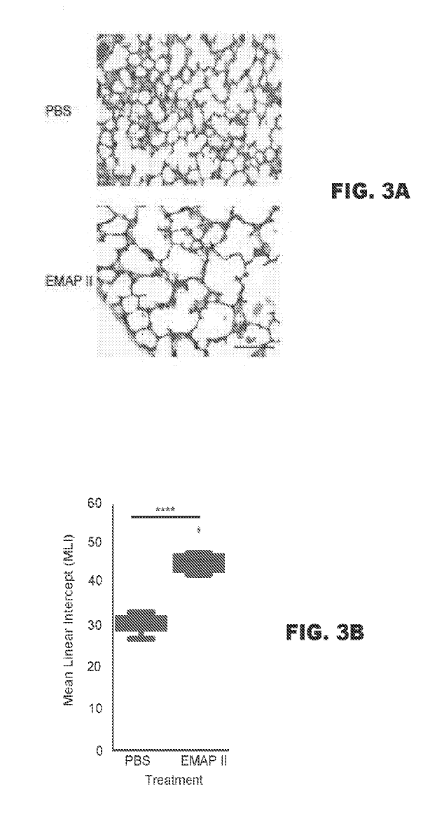

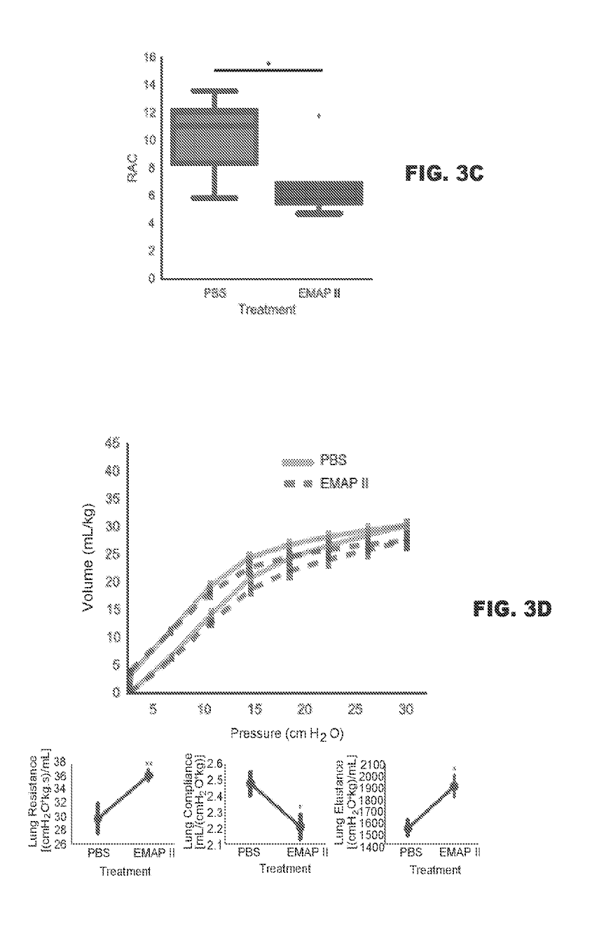

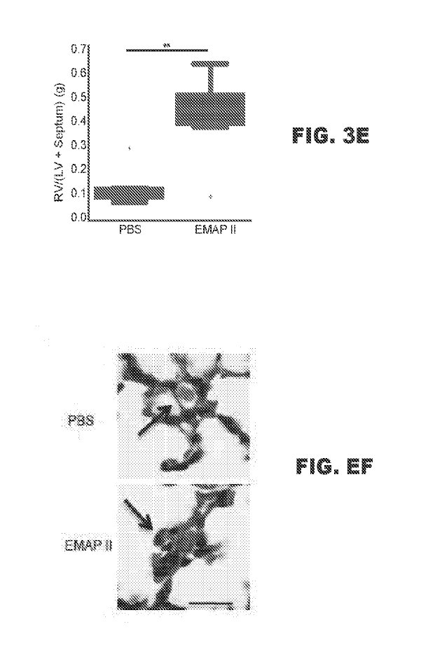

In addition to increased macrophage counts in mice administered EMAP II, we observed loss of lung structural integrity similar to that of BPD. As EMAP II has other reported functions, we sought out to define effects of sustained, elevated EMAP II on the lungs. Compared to control mice, the body weight of mice administered EMAP II was significantly lower, suggesting impaired overall growth (FIG. 8B). Lungs of EMAP II-administered mice had severely dysplastic alveoli and increased elastin deposition (FIG. 3A, F). There were larger distal airspaces as evidenced quantitatively by both significantly decreased radial alveolar count (RAC) and increased mean linear intercept (MLI) (FIG. 3B,C). This suggested that excess amounts of EMAP II impaired the lung structure. However, structure does not always correlate with lung function or outcome measurements (36).

Compared to control, mice given EMAP II had significantly impaired pulmonary biophysical properties. The pressure volume loop was shifted downward, suggesting an inability of lungs to maximally inflate along with other biophysical properties (FIG. 3D, FIG. 8C). To test whether impaired lung biophysical properties were due to surfactant expression, we measured surfactant protein-C (SP-C), a common indicator of type II alveolar epithelial cells that secrete surfactants. Compared to controls, mice administered EMAP II had significantly elevated mRNA and protein levels of SP-C (FIG. 7D, E).

This suggested a compensatory mechanism in response to exogenous EMAP II thus the lung function change was independent of a lack of SP-C.

EMAP II-Treated Mice Presented with Signs of Pulmonary Hypertension.

Macrophage counts were elevated following EMAP II injection, and previous studies link both the elevated counts and subsequent inflammatory cytokine release to pathogenesis of not only BPD but also its secondary sequel, pulmonary hypertension (PH) (37, 38).

Lungs of mice injected with EMAP II had impaired alveolarization and blood vessel formation leading to decreased function, reflecting anti-angiogenic properties of EMAP II (FIG. 3a); clinically, this implies cardiovascular sequelae, which are also prominent in poor outcome of BPD patients (7, 10). We observed right ventricular hypertrophy in mice given EMAP II compared to controls (FIG. 3E). Consistent with right-heart hypertrophy found in PH, we observed increased elastin deposition by Masson's Trichrome staining in distal vessels (FIG. 3F)

We concluded that chronic, elevated EMAP II led to BPD-like disease, including the development of signs of secondary PH. As SP-C levels were not decreased by EMAP II yet elevated EMAP II levels and macrophage recruitment were found in BPD, an alternative mechanism of upregulating EMAP II in early BPD must exist that modulates macrophage recruitment, negatively influencing lung and heart outcomes.

Neutralizing Excess EMAP II Limits Chemotactic Effects Upon Macrophages.

We tested if we could limit macrophage recruitment by neutralization of excess EMAP II. Using an EMAP II-neutralizing antibody (referred to as anti-EMAP II), we assessed macrophage transmigration in vitro. Consistent with the in vivo findings, we found that exogenous EMAP II significantly increased macrophage transmigration (FIG. 4A, B).

However, anti-EMAP II incubated with excess EMAP II significantly neutralized this chemoattraction in a dose-dependent manner (FIG. 4A, B). As a control, heat-inactivating EMAP II negated its function to increase transmigrated cells. Macrophage chemotaxis was specific to EMAP II and further confirmed by treating cells with LPS, an inflammatory agent with a role in macrophage migration and activation, and anti-EMAP II (FIG. 2A, B).

To assess whether we could prevent hyperoxia-induced BPD formation, mice were randomized and given the neutralizing anti-EMAP II antibody (FIG. 4C). Delivery of antibody to lungs was confirmed (FIG. 9A). Recruitment of macrophages in BPD mice was assessed by immunohistochemistry (FIG. 4D). Following treatment with anti-EMAP II, however, there was a significant decrease in the number of macrophages and inhibition of a BPD-like phenotype (FIG. 4E).

Neutralizing Excess EMAP II Improved Lung Structure and Development of Altered Function.

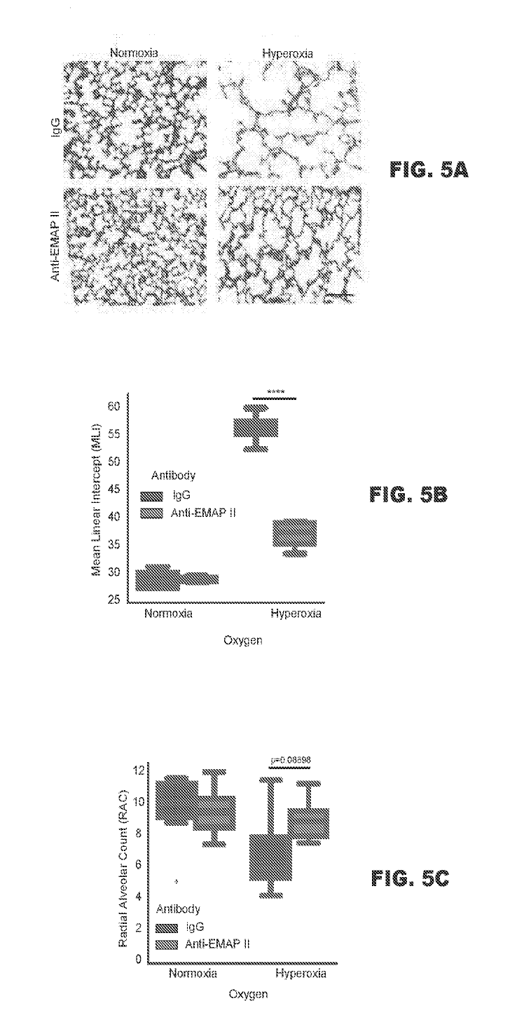

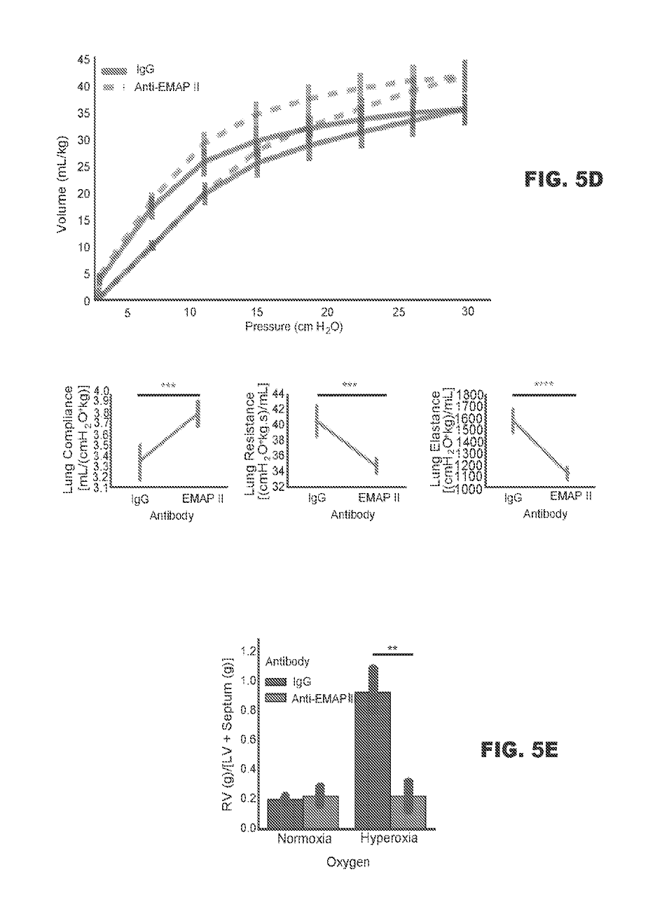

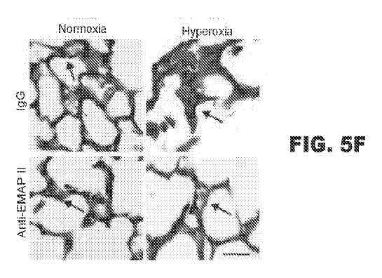

We considered that the inhibition of macrophages through neutralizing excess EMAP II in BPD would mitigate murine pulmonary damage. The body weight of hyperoxia mice treated with anti-EMAP II was comparable to control groups kept in room air (FIG. 9B). Following treatment (FIG. 4C), there was an increase in the number of distal alveoli measured in blinded manner, and a visible lack of bronchiolar vessel distension (FIG. 5A). As associated with parameters of the qualitative findings, there was a significant decrease in MLI counts, which reflects a decrease in empty air space (FIG. 5B). RAC counts in lungs of mice treated with anti-EMAP II appeared to increase compared to control non-specific IgG (FIG. 5C). By limiting macrophage recruitment, the hyperoxia mice treated with anti-EMAP II showed an improvement in pulmonary outcomes compared to mice treated with control IgG (FIG. 5D, FIG. 9E). There was a possibility that this improvement was not due to limiting macrophage recruitment but perhaps prevention of cellular apoptosis induced by either hyperoxia or EMAP II. We found increased apoptosis due to hyperoxia but an insignificant decrease following anti-EMAP II treatment (FIG. 9C). Another alternative mechanism would be an increase in surfactant production. SP-C did not significantly change following anti-EMAP II treatment, suggesting that the treatment was independent of surfactant production (FIG. 9D).

Anti-EMAP II Treatment Reduced Signs of PH.

To test whether anti-EMAP II treatment could impact development of PH, we assessed right ventricular hypertrophy. Significantly decreased right ventricular (RV) weight was seen in hearts of hyperoxia mice treated with anti-EMAP II over that of mice treated with control IgG, comparable to that of mice in room air (FIG. 5E). Consistent with right ventricular hypertrophy, we observed that there was elastin deposition in distal alveolar vessels (FIG. 5F).

Reducing Macrophage Numbers Resolved Inflammatory and Chemotactic Gene Expression.

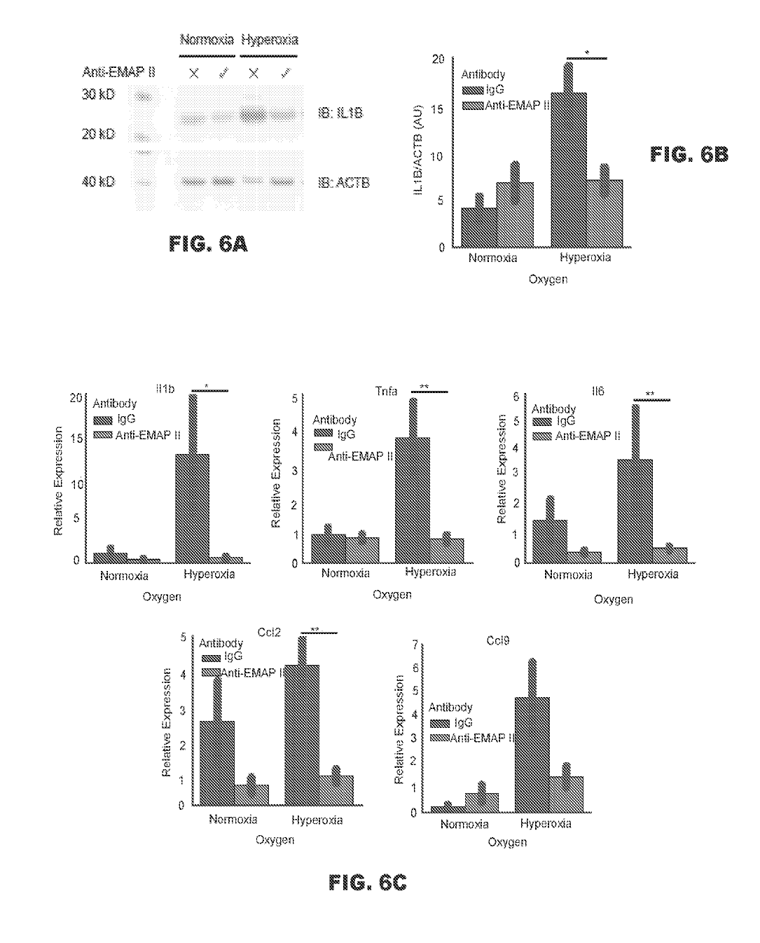

We proposed that by limiting macrophage recruitment through anti-EMAP II, we would reduce the levels of pro-inflammatory and chemotactic gene expression. By immunoblotting, we detected IL-1β levels in lungs of hyperoxia mice (FIG. 6A). Elevated IL-1β levels were significantly reduced in the hyperoxia mice treated with anti-EMAP II (FIG. 6B). In addition, expression of pro-inflammatory genes, Tnfa, Il6, Il1b and chemotactic genes, Ccl2, Ccl9 were markedly decreased following anti-EMAP II treatment (FIG. 6C).

Discussion

Premature birth, a major determinant of neonatal morbidity and mortality, is associated with long-term health consequences at an estimated expense of $26 billion per year in the United States alone. Lung disease of prematurity, BPD, is a preterm complication without a specific targeted treatment. After a call for more directed studies on pulmonary inflammation in BPD, clinical studies determined that inflammatory markers are not only elevated in BPD but associated with prognosis (12, 15, 39). Some studies used untargeted anti-inflammatory therapies such as glucocorticoids, direct cytokines, or chemokines with minimal improvement in some attributes of BPD (19).

In contrast, our results provide an opportunity to target pulmonary immune response by addressing macrophage infiltration as a therapeutic component of BPD. Our experiments show that EMAP II is a specific target that directly contributes to the pathogenesis of premature lung disease in BPD. This is manifested when elevated EMAP II was sustained in lungs of BPD mice compared to controls, corroborating the temporospatial-dependent role of EMAP II in BPD development of baboons and humans—specifically, in the bronchial epithelium rather than in perivasculature, where it is normally expressed and declines over time (31). In addition to sustained levels, the direct effect of EMAP II on BPD development was evident when mice treated with EMAP II developed a BPD-like phenotype: arrested alveolar development, right ventricular hypertrophy consistent with PH, macrophage recruitment, and heightened inflammatory state. Subsequently, anti-EMAP II treated mice in hyperoxia presented with a significant reduction in the inflammatory state and of the BPD-like phenotype.

The bronchial epithelium has recently been identified as the initial source of an immune response in various injury contexts (40, 41). Similarly, early marked elevated EMAP II expression in primary bronchial CCSP⁺ cells following hyperoxia support EMAP II's role as an inflammatory modulator in BPD development. Improvement in both macrophage counts and inflammation following anti-EMAP II treatment ascribes to its chemotactic function compared to its known anti-angiogenic function (21-23, 25, 29, 31, 42). Neutralization of EMAP II limited chemotaxis of macrophages in cell culture and into the lung, ultimately limiting inflammation. Given the proximal CCSP⁺ cell expression of EMAP II followed by macrophages expressing EMAP II in BPD mice, there exists a possible positive reinforcing cycle. Epithelial cells such as the CCSP⁺ cells express EMAP II, which recruits macrophages; these cells, in turn, can produce more EMAP II, which further propagates and activates other immune cells. If this is the case, a novel mechanism can be substantiated in clinical BPD development as a potential therapeutic target through the continuous presence of EMAP II.

Moving away from a simple dichotomy in macrophage activation reveals the many varying functional subsets in not only other disease contexts but also BPD. Two recent studies indicate that rather than a simple dichotomy in macrophage activation, a threshold of varying functional subsets of unknown origins (e.g. blood-derived circulating, bone marrow egression) is at least sufficient for BPD progression (19, 43). The first showed that elevated macrophage numbers in conjunction with pro-inflammatory gene expression resulted in BPD despite decreased counts of immune response cells (19). This suggests that a hyperactivated macrophage subset is crucial in hyperoxia-induced inflammation. The second study defined an alternative macrophage-like CD11b⁺ monocyte origin that protected BPD mice independent of neutrophilia (43). However, macrophage pro-inflammatory response is not only limited to lung disease of prematurity.

In agreement with the cited studies, our study shows significance in functional outcome dependent upon numbers of cells transduced by EMAP II. A previous study showed that elevated macrophage numbers in conjunction with pro-inflammatory gene expression resulted in BPD despite decreased counts of immune response cells (19). This suggests that hyperactivated macrophages are the major cell type in hyperoxia-induced inflammation. However, macrophage pro-inflammatory response is not only limited to lung disease of prematurity. Using this study of BPD as a working model the complex interactions of macrophages and their environment can be equally implicated as contributing or driving factors in other chronic inflammatory disease such as Crohn's disease or rheumatoid arthritis (44-46). Inhibition of excess macrophage numbers supports normal lung development, informing potential anti-inflammatory therapies.

In BPD, hyperoxia-induced inflammation has also been linked to impaired lung biophysical properties, but with conflicting results, as both increasing and decreasing compliance has been described (47, 48). Some studies suggest that hyperoxia increased compliance resembling emphysematous lungs, while other studies concluded hyperoxia decreased compliance due to the lungs being less pliable (47-49). We tested murine pulmonary outcomes at 6 weeks in concordance with previous studies (36, 47, 49).

Sustained EMAP II was associated with decreased compliance (FIG. 3D). For this reason, other biophysical properties, such as resistance, also need to be taken into account. Since impaired biophysical properties are collective, insufficient oxygen exchange, inflammation, and subsequent right ventricular hypertrophy contribute to pulmonary dysfunction. However, following anti-EMAP II treatment, vessels were not thickened, an indication of PH. Suppression of EMAP II inflammatory properties alleviated these pulmonary biophysical abnormalities associated with hyperoxia induced BPD including decreased resistance, decreased tissue damping, and decreased airway space.

Our results highlight an EMAP II-mediated inflammatory mechanism as a significant component of the multi-factorial pathogenesis of lung disease of prematurity, BPD. In contrast to other studies, the results of our experiments show not only robust protection from a BPD phenotype and signs of secondary PH but also reduction of macrophage recruitment and inflammatory status. Neutralization of EMAP II and curbing its ability to chemoattract macrophages is a possible future therapeutic goal in the prevention of BPD and secondary PH in the context of necessary chronic oxygen supplementation.

### REFERENCES

1. Blencowe H, Cousens S, Oestergaard M Z, Chou D, Moller A B, Narwal R, Adler A, Vera Garcia C, Rohde S, Say L, Lawn J E. National, regional, and worldwide estimates of preterm birth rates in the year 2010 with time trends since 1990 for selected countries: a systematic analysis and implications. Lancet 2012; 379: 2162-2172.
2. Liu L, Oza S, Hogan D, Perin J, Rudan I, Lawn J E, Cousens S, Mathers C, Black R E. Global, regional, and national causes of child mortality in 2000-13, with projections to inform post-2015 priorities: an updated systematic analysis. Lancet 2015; 385: 430-440.
3. Organization WH. Preterm Birth. [serial online] 2014. Available from: http://www.who.int/mediacentre/factsheets/fs363/en/
4. Stoll B J, Hansen N I, Bell E F, Walsh M C, Carlo W A, Shankaran S, Laptook A R, Sanchez P J, Van Meurs K P, Wyckoff M, Das A, Hale E C, Ball M B, Newman N S, Schibler K, Poindexter B B, Kennedy K A, Cotten C M, Watterberg K L, D'Angio C T, DeMauro S B, Truog W E, Devaskar U, Higgins R D, Eunice Kennedy Shriver National Institute of Child H, Human Development Neonatal Research N. Trends in Care Practices, Morbidity, and Mortality of Extremely Preterm Neonates, 1993-2012. JAMA 2015; 314: 1039-1051.
5. Bates M L, Farrell E T, Eldridge M W. Abnormal ventilatory responses in adults born prematurely. N Engl J Med 2014; 370: 584-585.
6. del Cerro M J, Sabate Rotes A, Carton A, Deiros L, Bret M, Cordeiro M, Verdu C, Barrios M I, Albajara L, Gutierrez-Larraya F. Pulmonary hypertension in bronchopulmonary dysplasia: clinical findings, cardiovascular anomalies and outcomes. Pediatr Pulmonol 2014; 49: 49-59.
7. Khemani E, McElhinney D B, Rhein L, Andrade O, Lacro R V, Thomas K C, Mullen M P. Pulmonary artery hypertension in formerly premature infants with bronchopulmonary dysplasia: clinical features and outcomes in the surfactant era. Pediatrics 2007; 120: 1260-1269.
8. Mourani P M, Sontag M K, Younoszai A, Miller J I, Kinsella J P, Baker C D, Poindexter B B, Ingram D A, Abman S H. Early pulmonary vascular disease in preterm infants at risk for bronchopulmonary dysplasia. Am J Respir Crit Care Med 2015; 191: 87-95.
9. Austin E D, Kawut S M, Gladwin M T, Abman S H. Pulmonary hypertension: NHLBI Workshop on the Primary Prevention of Chronic Lung Diseases. Ann Am Thorac Soc 2014; 11 Suppl 3: S178-185.
10. Bhat R, Salas A A, Foster C, Carlo W A, Ambalavanan N. Prospective analysis of pulmonary hypertension in extremely low birth weight infants. Pediatrics 2012; 129: e682-689.
11. Jobe A H, Ikegami M. Lung development and function in preterm infants in the surfactant treatment era. Annu Rev Physiol 2000; 62: 825-846.
12. McEvoy C T, Jain L, Schmidt B, Abman S, Bancalari E, Aschner J L. Bronchopulmonary dysplasia: NHLBI Workshop on the Primary Prevention of Chronic Lung Diseases. Ann Am Thorac Soc 2014; 11 Suppl 3: S146-153.
13. Desmarquest P, Chadelat K, Corroyer S, Cazals V, Clement A. Effect of hyperoxia on human macrophage cytokine response. Respir Med 1998; 92: 951-960.
14. Eriksson L, Haglund B, Odlind V, Altman M, Kieler H. Prenatal inflammatory risk factors for development of bronchopulmonary dysplasia. Pediatr Pulmonol 2014; 49: 665-672.
15. Ambalavanan N, Carlo W A, D'Angio C T, McDonald S A, Das A, Schendel D, Thorsen P, Higgins R D, Eunice Kennedy Shriver National Institute of Child H, Human Development Neonatal Research N. Cytokines associated with bronchopulmonary dysplasia or death in extremely low birth weight infants. Pediatrics 2009; 123: 1132-1141.
16. Abraham E, Anzueto A, Gutierrez G, Tessler S, San Pedro G, Wunderink R, Dal Nogare A, Nasraway S, Berman S, Cooney R, Levy H, Baughman R, Rumbak M, Light R B, Poole L, Allred R, Constant J, Pennington J, Porter S. Double-blind randomised controlled trial of monoclonal antibody to human tumour necrosis factor in treatment of septic shock. NORASEPT II Study Group. Lancet 1998; 351: 929-933.
17. Opal S M, Fisher C J, Jr., Dhainaut J F, Vincent J L, Brase R, Lowry S F, Sadoff J C, Slotman G J, Levy H, Balk R A, Shelly M P, Pribble J P, LaBrecque J F, Lookabaugh J, Donovan H, Dubin H, Baughman R, Norman J, DeMaria E, Matzel K, Abraham E, Seneff M. Confirmatory interleukin-1 receptor antagonist trial in severe sepsis: a phase III, randomized, double-blind, placebo-controlled, multicenter trial. The Interleukin-1 Receptor Antagonist Sepsis Investigator Group. Crit Care Med 1997; 25: 1115-1124.
18. Oncel M Y, Yurttutan S, Alyamac Dizdar E, Gokce I K, Gonul, I I, Topal T, Canpolat F E, Dilmen U. Beneficial Effect of Etanercept on Hyperoxic Lung Injury Model in Neonatal Rats. J Invest Surg 2015: 1-5.
19. Nold M F, Mangan N E, Rudloff I, Cho S X, Shariatian N, Samarasinghe T D, Skuza E M, Pedersen J, Veldman A, Berger P J, Nold-Petry C A. Interleukin-1 receptor antagonist prevents murine bronchopulmonary dysplasia induced by perinatal inflammation and hyperoxia. Proc Natl Acad Sci USA 2013; 110: 14384-14389.
20. Johnson B H, Yi M, Masood A, Belcastro R, Li J, Shek S, Kantores C, Jankov R P, Tanswell A K. A critical role for the IL-1 receptor in lung injury induced in neonatal rats by 60% O2. Pediatr Res 2009; 66: 260-265.
21. Schwarz M A, Zhang F, Gebb S, Starnes V, Warburton D. Endothelial monocyte activating polypeptide II inhibits lung neovascularization and airway epithelial morphogenesis. Mech Dev 2000; 95: 123-132.
22. Schwarz M A, Kandel J, Brett J, Li J, Hayward J, Schwarz R E, Chappey O, Wautier J L, Chabot J, Lo Gerfo P, Stern D. Endothelial-monocyte activating polypeptide II, a novel antitumor cytokine that suppresses primary and metastatic tumor growth and induces apoptosis in growing endothelial cells. J Exp Med 1999; 190: 341-354.
23. Reznikov A G, Chaykovskaya L V, Polyakova L I, Kornelyuk A I. Antitumor effect of endothelial monocyte-activating polypeptide-II on human prostate adenocarcinoma in mouse xenograft model. Exp Oncol 2007; 29: 267-271.
24. Tsai B M, Wang M, Clauss M, Sun P, Meldrum D R. Endothelial monocyte-activating polypeptide II causes NOS-dependent pulmonary artery vasodilation: a novel effect for a proinflammatory cytokine. Am J Physiol Regul Integr Comp Physiol 2004; 287: R767-771.
25. Clauss M, Voswinckel R, Rajashekhar G, Sigua N L, Fehrenbach H, Rush N I, Schweitzer K S, Yildirim A O, Kamocki K, Fisher A J, Gu Y, Safadi B, Nikam S, Hubbard W C, Tuder R M, Twigg H L, 3rd, Presson R G, Sethi S, Petrache I. Lung endothelial monocyte-activating protein 2 is a mediator of cigarette smoke-induced emphysema in mice. J Clin Invest 2011; 121: 2470-2479.
26. van Horssen R, Rens J A, Brunstein F, Guns V, van Gils M, Hagen T L, Eggermont A M. Intratumoural expression of TNF-R1 and EMAP-II in relation to response of patients treated with TNF-based isolated limb perfusion. Int J Cancer 2006; 119: 1481-1490.
27. Schikorski D, Cuvillier-Hot V, Boidin-Wichlacz C, Slomianny C, Salzet M, Tasiemski A. Deciphering the immune function and regulation by a TLR of the cytokine EMAPII in the lesioned central nervous system using a leech model. J Immunol 2009; 183: 7119-7128.
28. Clarijs R, Schalkwijk L, Ruiter D J, de Waal R M. EMAP-II expression is associated with macrophage accumulation in primary uveal melanoma. Invest Ophthalmol Vis Sci 2003; 44: 1801-1806.
29. Schwarz M, Lee M, Zhang F, Zhao J, Jin Y, Smith S, Bhuva J, Stern D, Warburton D, Starnes V. EMAP II: a modulator of neovascularization in the developing lung. Am J Physiol 1999; 276: L365-375.
30. Zhang F, Schwarz M A. Temporo-spatial distribution of endothelial-monocyte activating polypeptide II, an anti-angiogenic protein, in the mouse embryo. Dev Dyn 2000; 218: 490-498.
31. Quintos-Alagheband M L, White C W, Schwarz M A. Potential role for antiangiogenic proteins in the evolution of bronchopulmonary dysplasia. Antioxid Redox Signal 2004; 6: 137-145.
32. Lee D D, Schwarz M A. Adapted approach to profile genes while reconciling Vegf-a mRNA expression in the developing and injured lung. Am J Physiol Lung Cell Mol Physiol 2015; 308: L1202-1211.
33. Bennett K M, Afanador M D, Lal C V, Xu H, Persad E, Legan S K, Chenaux G, Dellinger M, Savani R C, Dravis C, Henkemeyer M, Schwarz M A. Ephrin-B2 reverse signaling increases alpha5beta1 integrin-mediated fibronectin deposition and reduces distal lung compliance. Am J Respir Cell Mol Biol 2013; 49: 680-687.
34. Nuhrenberg T G, Langwieser N, Schwarz J B, Hou Y, Frank P, Sorge F, Matschurat S, Seidl S, Kastrati A, Schomig A, Clauss M A, Zohlnhofer D. EMAP-II downregulation contributes to the beneficial effects of rapamycin after vascular injury. Cardiovasc Res 2008; 77: 580-589.
35. Schwarz R E, Schwarz M A. In vivo therapy of local tumor progression by targeting vascular endothelium with EMAP-II. J Surg Res 2004; 120: 64-72.
36. Manali E D, Moschos C, Triantafillidou C, Kotanidou A, Psallidas I, Karabela S P, Roussos C, Papiris S, Armaganidis A, Stathopoulos G T, Maniatis N A. Static and dynamic mechanics of the murine lung after intratracheal bleomycin. BMC Pulm Med 2011; 11: 33.
37. Jankov R P, Luo X, Belcastro R, Copland I, Frndova H, Lye S J, Hoidal J R, Post M, Tanswell A K. Gadolinium chloride inhibits pulmonary macrophage influx and prevents O(2)-induced pulmonary hypertension in the neonatal rat. Pediatr Res 2001; 50: 172-183.
38. Masood A, Yi M, Lau M, Belcastro R, Li J, Kantores C, Pace-Asciak C R, Jankov R P, Tanswell A K. Cyclooxygenase-2 inhibition partially protects against 60% O2-mediated lung injury in neonatal rats. Pediatr Pulmonol 2014; 49: 991-1002.
39. Walsh M C, Szefler S, Davis J, Allen M, Van Marter L, Abman S, Blackmon L, Jobe A. Summary proceedings from the bronchopulmonary dysplasia group. Pediatrics 2006; 117: S52-56.
40. Evans C M, Williams O W, Tuvim M J, Nigam R, Mixides G P, Blackburn M R, DeMayo F J, Burns A R, Smith C, Reynolds S D, Stripp B R, Dickey B F. Mucin is produced by clara cells in the proximal airways of antigen-challenged mice. Am J Respir Cell Mol Biol 2004; 31: 382-394.
41. Gibbs J, Ince L, Matthews L, Mei J, Bell T, Yang N, Saer B, Begley N, Poolman T, Pariollaud M, Farrow S, DeMayo F, Hussell T, Worthen G S, Ray D, Loudon A. An epithelial circadian clock controls pulmonary inflammation and glucocorticoid action. Nat Med 2014; 20: 919-926.
42. Yuan C, Yan L, Solanki P, Vatner S F, Vatner D E, Schwarz M A. Blockade of EMAP II protects cardiac function after chronic myocardial infarction by inducing angiogenesis. J Mol Cell Cardiol 2015; 79: 224-231.
43. Eldredge L C, Treuting P M, Manicone A M, Ziegler S F, Parks W C, McGuire J K. CD11b(+) Mononuclear Cells Mitigate Hyperoxia-Induced Lung Injury in Neonatal Mice. Am J Respir Cell Mol Biol 2016; 54: 273-283.
44. Thiesen S, Janciauskiene S, Uronen-Hansson H, Agace W, Hogerkorp C M, Spee P, Hakansson K, Grip 0. CD14(hi)HLA-DR(dim) macrophages, with a resemblance to classical blood monocytes, dominate inflamed mucosa in Crohn's disease. J Leukoc Biol 2014; 95: 531-541.
45. Murthy A, Li Y, Peng I, Reichelt M, Katakam A K, Noubade R, Roose-Girma M, DeVoss J, Diehl L, Graham R R, van Lookeren Campagne M. A Crohn's disease variant in Atg16l1 enhances its degradation by caspase 3. Nature 2014; 506: 456-462.
46. Vande Walle L, Van Opdenbosch N, Jacques P, Fossoul A, Verheugen E, Vogel P, Beyaert R, Elewaut D, Kanneganti T D, van Loo G, Lamkanfi M. Negative regulation of the NLRP3 inflammasome by A20 protects against arthritis. Nature 2014; 512: 69-73.
47. Yee M, Chess P R, McGrath-Morrow S A, Wang Z, Gelein R, Zhou R, Dean D A, Notter R H, O'Reilly M A. Neonatal oxygen adversely affects lung function in adult mice without altering surfactant composition or activity. Am J Physiol Lung Cell Mol Physiol 2009; 297: L641-649.
48. Yee M, White R J, Awad H A, Bates W A, McGrath-Morrow S A, O'Reilly M A. Neonatal hyperoxia causes pulmonary vascular disease and shortens life span in aging mice. Am J Pathol 2011; 178: 2601-2610.
49. Woyda K, Koebrich S, Reiss I, Rudloff S, Pullamsetti S S, Ruhlmann A, Weissmann N, Ghofrani H A, Gunther A, Seeger W, Grimminger F, Morty R E, Schermuly R T. Inhibition of phosphodiesterase 4 enhances lung alveolarisation in neonatal mice exposed to hyperoxia. Eur Respir J 2009; 33: 861-870.
50. Ramani M, Bradley W E, Dell'Italia L J, Ambalavanan N. Early exposure to hyperoxia or hypoxia adversely impacts cardiopulmonary development. Am J Respir Cell Mol Biol 2015; 52: 594-602.
51. Betz P, Nerlich A, Bussler J, Hausmann R, Eisenmenger W. Radial alveolar count as a tool for the estimation of fetal age. Int J Legal Med 1997; 110: 52-54.

Supplementary Methods

### Cell Lines and Reagents

RAW264.7 cell line was obtained from ATCC. The EMAP II neutralizing antibody was developed in-house, raised against a 13 peptide sequence of the C-terminus portion—the dosage and timing determined by previous clearance studies (data not shown). Its efficacy on known anti-angiogenic effect was demonstrated in a myocardial infarction model (42). LPS derived from Serotype 055:B5 E. coli (L6529, Sigma-Aldrich).

### Recombinant EMAP II Preparation

6×-His tagged EMAP II was prepared as previously described (21). Endotoxin concentration in samples was <0.1 EU/mL as determined by LAL assay (88282, Pierce).

### Mice

C57BL/6 mice were obtained from Jackson Laboratories. Studies using mice were performed according to the animal protocols approved by the Indiana University Institutional Animal Care and Use Committee.

### Mouse Model of Bronchopulmonary Dysplasia and Therapy

Newborn pups were randomly selected to receive either rabbit non-specific immunoglobulin G or rabbit anti-EMAP II neutralizing antibody (2 mg/kg) as illustrated in FIG. 4c.

Bronchopulmonary dysplasia during alveologenesis was then modeled and illustrated in the above figure(50). To collect tracheal aspirates from mice, a small incision was made along with the anterior portion of the trachea. An angiocatheter was then inserted within the tracheal incision. Two hundred microliters of fluid was inserted and aspirated. Fluid was then centrifuged at 5,000 g for 5 minutes at 4° C.

EMAP II Treatment of Mice

Mice were subcutaneously injected with recombinant EMAP II (80 μg/kg in 100 μL) daily beginning from day 3 to day 14 as shown in FIG. 2a. This dosage had been previously established as having an anti-angiogenic effect (35).

Immunoblotting

Sections of the right lung were placed in tubes containing modified lysis buffer. The tubes were then placed and homogenized in Bullet Blender Storm (BBY24M, Next Advance Inc.). Protein concentrations were determined by Bradford reagent (Bio-Rad). Twenty micrograms of lung tissue homogenates were loaded into NuPage Novex 4-12% Bis-Tris Protein gels (Invitrogen), incubated in 20% ethanol for 5 minutes, and transferred onto nitrocellulose membranes using iBlot v2 (Invitrogen). The membranes were placed in 5% blocking buffer (BioRad) in tris-buffered saline with 0.05% Tween-20 for 1 hour at room temperature. Membranes were incubated overnight at 4° C. in primary antibodies listed in Supplementary Table 1.

Lung Microscopy

Lung tissues were inflation fixed by dripping 4% paraformaldehyde (w/v in PBS) at a height of 25 cm mm.H₂O above the lung for 10-15 minutes. Lungs were excised from the mouse en bloc and placed in 4% paraformaldehyde overnight before embedding in paraffin. Embedded tissues were sectioned 5.0 μm thick (Zeiss). Antigen retrieval and antibody staining was performed according to Supplementary Table 1. Hematoxylin and eosin staining and Masson's Trichrome staining was performed according to manufacturer's instructions (Thermo Fisher). Co-localization staining was performed using Enzymatic double-staining IHC kit (Abcam). Images were captured on Hammamatsu Orca-ER or DP70 camera at magnifications indicated in the appropriate figure legends using CellSens software.

Lung Morphometry Analysis

Mean linear intercepts and radial alveolar counts were calculated from H&E stained lung sections counted blind, decoded(51).

Pulmonary Function Testing

7Only male mice were tested for pulmonary functions to avoid possible hormonal issues. Mice were anesthetized with ketamine (100 mg/kg) and xylazine (6 mg/kg) followed by pancuronium (1 mg/kg) to induce paralysis. A metal cannulus was inserted through a small incision in the trachea followed by single-model and complex model measurements of lung function using FlexiVent Software (SCIREQ Inc.).

Macrophage Count

Light microscopy images were taken on Olympus using MicroBiological Suites with a 40× objective lens. The images were then randomized and the number of GAL-3 positive cells were counted blinded. The images were then decoded and analyzed using Python.

Quantitative PCR

Lung harvest, RNA extraction, RNA quality determination, quantitative PCR, and analysis were performed according to the methods in a previous study⁽³²⁾. Primers are listed in Supplementary Table 1.

Transmigration Assay

AW264.7 cells were cultured in phenol-red free DMEM media containing 10% FBS, antimicrobial and antifungal supplement, 5 mM HEPES, and 5 mM L-Glutamine until approximately 70-80% confluency. The media was then exchanged for transmigration media which contained phenol-red free DMEM containing 1% FBS for 2 hours before being scraped, incubated in CD16/32 to block non-specific F′ab interactions on ice for approximately 15 minutes; the media containing the antibody was centrifuged at 400×g for 5 minutes at 4° C. and aspirated. 5×10⁴ cells were resuspended in transmigration media and loaded into a single 5.0-micron pore transwell insert. Regarding treatments in FIG. 4, EMAP II protein was either boiled for 30 minutes at 100° C. or pre-incubated with EMAP II neutralizing antibody at room temperature for 30 minutes at respective dosages. LPS (Serotype E. Coli 055:B4, Sigma) was also pre-incubated with EMAP II neutralizing antibody. The bottom inserts were filled with 500 microliters containing the listed treatments. Transmigration occurred for 4 hours at 37° C., fixed in 4% paraformaldehyde (w/v in PBS) overnight, and stained in crystal violet solution. Images were captured at 20× magnification on DP70 using MicroSuite Biological Software. n=4-6 for each treatment Outliers. Data that were statistically significant by Grubb's Outlier test were removed from analysis.

SUPPLEMENTARY TABLE 1

| Target | Sense | Antisense | Length |
|---|---|---|---|
| Hprt | CCCCAAAATGGTTAAGGTTG | AACAAAGTCTGGCCTGTAT | 76 |
| | C (SEQ ID NO: 1) | CC (SEQ ID NO: 2) | |
| Eefl | ACATTCTCACCGACATCACC | GAACATCAAACCGCACACC | 135 |
| | (SEQ ID NO: 3) | (SEQ ID NO: 4) | |
| Rp113a | TCCCTCCACCCTATGACAAG | GTCACTGCCTGGTACTTCC | 136 |
| | (SEQ ID NO:5) | (SEQ ID NO: 6) | |
| Kdr | GTACCGGGACGTCGACATAG | GTACCGGGACGTCGACATA | 79 |
| | (SEQ ED NO: 7) | G (SEQ ID NO: 8) | |
| Flt1 | ACTCTTGTCCTCAACTGCAC | GGTCAATCCGCTGCCTTAT | 112 |
| | (SEQ ID NO: 9) | AG (SEQ ID NO: 10) | |
| Sftpc | ATGGACATGAGTAGCAAAGA | CACGATGAGAAGGCGTTTG | |
| | GGT (SEQ ID NO: 11) | AG (SEQ ID NO: 12) | |

| Target | Assay ID | Vendor |
|---|---|---|
| Il1b | qMmuCID0005641 | BioRad |
| Tnf | qMmuCED0004141 | BioRad |
| Il6 | qMmuCID0005613 | BioRad |
| Ccl2 | qMmuCEP0056726 | BioRad |
| Ccl9 | qMmuCID0021820 | BioRad |

Antibody Used

| | | | Catalog | | | |
|---|---|---|---|---|---|---|
| Antigen | Clone | Company | No. | Usage | Antigen Retrieval | Dilution |
| IL1B | 3A6 | Cell Signalling | 12242S | IB | — | 1:1000 |
| ACTB | 8H10D10, | Cell Signalling | 3700S, | IB | — | 1:10,000, |
| | C4 | EMD | MAB15 | | | 1:2000 |
| | | Millipore | 01 | | | |
| SPC | — | EMD | AB3786 | IB, IHC | None | 1:1000 |
| | | Millipore | | | | |
| GAL-3 | M3-38 | Antibodies- | ABIN18 | IHC | Citrate | 1:1000 |
| | | Online | 04652 | | | |
| CCSP | — | Seven Hills | WRAB- | IHC | Citrate | 1:1000 |
| | | | 3950 | | | |
| CD16/32 | 93 | eBioscience | 130- | Transwell | | |
| | | | 0161-82 | | | |
| EMAP | — | In-house | — | IB, IHC, | Trypsin | 1:5000, |
| II | | | | Transwell | | 1:100 |

IB: Immunoblot
IHC: Immunohistochemistry

It is to be understood, however, that these examples are provided by way of illustration and nothing herein should be taken as a limitation upon the overall scope of the invention.

1. A pharmaceutical composition for treatment of a lung condition in a subject comprising
(a) a therapeutically effective amount of an antagonist of Endothelial Monocyte-Activating Polypeptide II (EMAP II) and
(b) a pharmaceutically suitable carrier.

2. The composition of claim 1, wherein the lung condition is bronchopulmonary dysplasia (BPD).

3. The composition of claim 1, wherein the antagonist of EMAP II is selected from the group consisting of an anti-EMAP II antibody, an antibody specific for an EMAP II receptor, and a soluble EMAP II receptor.

4. The pharmaceutical composition of claim 1, wherein the pharmaceutical composition is formulated for administration intraveneously, paraterally, orally, topically or by aerosol.

5. The pharmaceutical composition of claim 3, wherein the pharmaceutical composition is formulated for inhalation administration.

6. A method of treating a lung condition in an infant in need thereof, the method comprising administering to the subject a therapeutically effective amount of the pharmaceutical composition of claim 1, whereby the lung condition is treated.

7. The method of claim 6, wherein the lung condition is bronchopulmonary dysplasia, and wherein the pharmaceutical composition is used to ameliorate bronchopulmonary dysplasia in a subject that has been diagnosed with the disease.

8. The method of claim 7, wherein the subject suffers from secondary pulmonary hypertension associated with bronchopulmonary dysplasia.

9. The method of claim 6, further comprising administering at least one additional agent or therapy selected from the group consisting of a surfactant, oxygen therapy, ventilator therapy, steroid, or inhaled nitric oxide.

10. The method of claim 6, wherein the pharmaceutical composition is administered by intraveneous, parenteral, oral, or by aerosol.

11. The method of claim 10, wherein the pharmaceutical composition is administered by aerosol.

12. The method of claim 6, wherein the subject is an infant.

13. The method of claim 12, wherein the infant is a neonate.

14. A method of treating an infant at risk of developing bronchopulmonary dysplasia (BPD) comprising administering a therapeutically effective amount of the pharmaceutical composition of claim 1 to the infant.

15. The method of claim 14, further comprising administering at least one additional agent or therapy selected from the group consisting of a surfactant, oxygen therapy, ventilator therapy, steroid, or inhaled nitric oxide.

16. The method of claim 14, wherein the infant is a neonate.

17. The method of claim 14, wherein the pharmaceutical composition is administered by intravenous, parenteral, oral, or aerosol.

18. The method of claim 17, where the pharmaceutical composition is formulated for aerosol delivery.

19. A method of reducing macrophage infiltration into the lungs of a subject suffering from bronchopulmonary dysplasia, the method comprising administering a therapeutically effective amount of the pharmaceutical composition of claim 1 to reduce the number of macrophage infiltrating into the lung of the subject.

20. The method of claim 19, wherein the subject is an infant.

21. The method of claim 19, further comprising administering at least one additional agent or therapy selected from the group consisting of a surfactant, oxygen therapy, ventilator therapy, steroid, or inhaled nitric oxide.

22-28. (canceled)


SEQUENCE LISTING (13 sequences)

SEQ ID NO: 1 - DNA, 21 bp, Artificial Sequence
  synthetic
ccccaaaatggttaaggttgc

SEQ ID NO: 2 - DNA, 21 bp, Artificial Sequence
  synthetic
aacaaagtctggcctgtatcc

SEQ ID NO: 3 - DNA, 20 bp, Artificial Sequence
  synthetic
acattctcaccgacatcacc

SEQ ID NO: 4 - DNA, 19 bp, Artificial Sequence
  synthetic
gaacatcaaaccgcacacc

SEQ ID NO: 5 - DNA, 20 bp, Artificial Sequence
  synthetic
tccctccaccctatgacaag

SEQ ID NO: 6 - DNA, 19 bp, Artificial Sequence
  synthetic
gtcactgcctggtacttcc

SEQ ID NO: 7 - DNA, 20 bp, Artificial Sequence
  synthetic
gtaccgggacgtcgacatag

SEQ ID NO: 8 - DNA, 20 bp, Artificial Sequence
  synthetic
gtaccgggacgtcgacatag

SEQ ID NO: 9 - DNA, 20 bp, Artificial Sequence
  synthetic
actcttgtcctcaactgcac

SEQ ID NO: 10 - DNA, 21 bp, Artificial Sequence
  synthetic
ggtcaatccgctgccttatag

SEQ ID NO: 11 - DNA, 23 bp, Artificial Sequence
  syntehtic
atggacatgagtagcaaagaggt

SEQ ID NO: 12 - DNA, 21 bp, Artificial Sequence
  synthetic
cacgatgagaaggcgtttgag

SEQ ID NO: 13 - protein, 13 aa, Artificial Sequence
  synthetic
DAFPGEPDKELNP